(19) United States
(12) Patent Application Publication
(10) Pub. No.: US 2024/0122918 A1
(43) Pub. Date: Apr. 18, 2024

(54) NanoMesh Drug Delivery for Treatment of Brain Tumors

(71) Applicants: University of Cincinnati, Cincinnati, OH (US); The Johns Hopkins University, Baltimore, MD (US)

(72) Inventors: Andrew Steckl, Cincinnati, OH (US); Daewoo Han, Cincinnati, OH (US); Betty M. Tyler, Baltimore, MD (US); Henry Brem, Baltimore, MD (US); Safwan Alomari, Baltimore, MD (US)

(21) Appl. No.: 18/380,984

(22) Filed: Oct. 17, 2023

Related U.S. Application Data
(60) Provisional application No. 63/416,785, filed on Oct. 17, 2022.

(51) Int. Cl. A61K 31/473; A61K 9/70; A61K 31/277; A61K 31/4188; A61K 47/34; B23K 26/38; B32B 5/02; B32B 5/26; B32B 37/10; B32B 37/18; B32B 38/00; B32B 38/18; D04H 1/435; D04H 1/4382; D04H 1/56; D04H 1/728; D06C 15/00

(52) U.S. Cl. CPC A61K 31/473; A61K 9/7007; A61K 31/277; A61K 31/4188; A61K 47/34; B23K 26/38; B32B 5/022; B32B 5/266; B32B 37/1045; B32B 37/18; B32B 38/0004; B32B 38/0008; B32B 38/18; D04H 1/435; D04H 1/43828; D04H 1/43838; D04H 1/56; D04H 1/728; D06C 15/00; B32B 2250/20; B32B 2255/02; B32B 2255/26; B32B 2262/0276; B32B 2262/124; B32B 2305/28; B32B 2309/02; B32B 2310/0843; B32B 2367/00; B32B 2535/00; D10B 2331/041; D10B 2331/30; D10B 2509/00

(57) ABSTRACT

A drug delivery device comprising: a first layer comprising a first coaxially electrospun nanofiber membrane; a second layer comprising a second coaxially electrospun nanofiber membrane; a first therapeutic agent integrated into the first coaxially electrospun nanofiber membrane; and a second therapeutic agent integrated into the second coaxially electrospun nanofiber membrane. The second therapeutic agent is different from the first therapeutic agent.

### CROSS REFERENCE TO RELATED APPLICATION

This application claims priority to U.S. Provisional Application Ser. No. 63/416,785, filed Oct. 17, 2022, the entire contents of which are incorporated herein by reference.

### STATEMENT REGARDING FEDERALLY SPONSORED RESEARCH OR DEVELOPMENT

This invention was made with government support under R15 CA252987 awarded by the National Institutes of Health. The government has certain rights in the invention.

### TECHNICAL FIELD

The present disclosure relates to the field of drug delivery devices. Specifically, the present disclosure relates to multi-layered electrospun nanofiber structures for use in the delivery of drugs.

### BACKGROUND

Glioblastoma multiforme (GBM) is the most common and aggressive primary brain neoplasm in adults. Due to the presence of the GBM stem cell population, as well as high tumor heterogeneity, proliferative rate, infiltrative nature, and marked vascularity, available therapies have increased median survival to only a modest 15 months. Interstitial chemotherapy (e.g., carmustine implant, marketed under the brand name Gliadel®) has played a pivotal role in the treatment of GBM by enhancing drug biodistribution to the tumor bed and minimizing systemic toxicities. However, Gliadel® has yielded only modest statistical significance in the prolongation of patient survival due to two main reasons. First, the drug-loading dose of Gliadel® is relatively low. Second, due to the GBM heterogeneity to chemotherapy, there is a compelling need to use multiple therapeutics with complementary mechanisms to limit the tumor resistance.

Studies have demonstrated that tumor hypoxia is strongly correlated with GBM stem cell phenotype, immune microenvironment, neo-angiogenesis, invasive capacity of the tumor, as well as tumor recurrence, resistance to chemotherapy and radiotherapy, and decreased survival rates. These pathways rely on hypoxia-inducible factors (e.g., HIF-1α and HIF-2α) and their downstream cascades. HIF-1α promotes angiogenesis and regulates metabolic pathways. HIF-2α has been found to be selectively expressed in tumor cells and plays a key role in stem cell phenotype expression. Both HIF-1α and HIF-2α have shown prognostic implications, with higher expression in tumors correlating with increased tumor grade and poor patient survival. Local delivery of the combination of acriflavine (ACF, HIF-1α inhibitor), PT-2385 (HIF-2α inhibitor), and temozolomide (TMZ. FDA-approved alkylating agent) has demonstrated impressive results in patient-derived xenograft (PDX) models. Each of these three drugs targets a major tumor cascade and together have complimentary mechanisms of action that can lead to a direct intra-tumor cytotoxicity and an indirect anti-tumor immune response.

A need exists for improved delivery of drugs to therapeutic targets, including GBM tumor cavities.

### SUMMARY

The present disclosure is directed to practical, easy-to-handle, and robust drug delivery devices that are formed as a densified multi-layer structure containing coaxially electrospun nanofiber membranes. Importantly, by selectively stacking certain membranes, programmable sequential and/or simultaneous release of one or multiple drugs can be attained by the drug delivery devices of the present disclosure. Additionally, the drug delivery devices of the present disclosure permit simultaneous release of multiple drugs from coaxially electrospun nanofibers that form the membranes. Without wishing to be bound by theory, the drug delivery devices presented herein are expected to demonstrate controlled and sustained delivery of multiple anti-cancer agents, such as ACF (an HIF-1α inhibitor) and PT2385 (an HIF-2α inhibitor), to the tumor bed.

According to a first aspect of the present disclosure, a drug delivery device comprises: a first layer comprising a first coaxially electrospun nanofiber membrane; a second layer comprising a second coaxially electrospun nanofiber membrane; a first therapeutic agent integrated into the first coaxially electrospun nanofiber membrane; and a second therapeutic agent integrated into the second coaxially electrospun nanofiber membrane. The second therapeutic agent is different from the first therapeutic agent.

A second aspect may include the first aspect, wherein the first and second therapeutic agents are anti-neoplastic drugs.

A third aspect may include any one of the first or second aspects, wherein the first therapeutic agent is configured to inhibit a first hypoxia-inducible factor and the second therapeutic agent is an alkylating agent.

A fourth aspect may include the third aspect, further comprising a third therapeutic agent integrated into the first coaxially electrospun nanofiber membrane and configured to inhibit a second hypoxia-inducible factor different from the first hypoxia-inducible factor.

A fifth aspect may include the fourth aspect, wherein: the first hypoxia-inducible factor is HIF-1α; and the second hypoxia-inducible factor is HIF-2α.

A sixth aspect may include the fifth aspect, wherein: the first therapeutic agent is acriflavine (ACF); the third therapeutic agent is PT2385; and the alkylating agent is temozolomide (TMZ).

A seventh aspect may include any one of the first through sixth aspects, wherein: the first coaxially electrospun nanofiber membrane is folded and compressed to form a first coaxially electrospun nanofiber NanoMesh; the second coaxially electrospun nanofiber membrane is folded and compressed to form a second coaxially electrospun nanofiber NanoMesh; the first layer comprises a first disc that has been laser cut from the first coaxially electrospun nanofiber NanoMesh; and the second layer comprises a second disc that has been laser cut from the second coaxially electrospun nanofiber NanoMesh.

An eighth aspect may include any one of the first through seventh aspects, further comprising a third layer comprising a third coaxially electrospun nanofiber membrane, wherein the second therapeutic agent is also incorporated into the third coaxially electrospun nanofiber membrane, and wherein: the second layer is positioned outward from a first surface of the first layer; and the third layer is positioned outward from a second surface of the first layer, wherein the second surface of the first layer is opposite the first surface of the first layer.

A ninth aspect may include the eighth aspect, further comprising: a first intermediate layer interposed between the first layer and the second layer; and a second intermediate layer interposed between the first layer and the third layer.

A tenth aspect may include the ninth aspect, wherein the first and second intermediate layers are hydrophobic.

An eleventh aspect may include any one of the ninth or tenth aspects, wherein each of the first and second intermediate layers comprises an electrospun nanofiber membrane.

A twelfth aspect may include any one of the ninth through eleventh aspects, further comprising a third therapeutic agent integrated into the first coaxially electrospun nanofiber membrane, wherein: the first therapeutic agent is configured to inhibit a first hypoxia-inducible factor; and the third therapeutic agent is configured to inhibit a second hypoxia-inducible factor different from the first hypoxia-inducible factor.

A thirteenth aspect may include the twelfth aspect, wherein: the first hypoxia-inducible factor is HIF-1α; the second hypoxia-inducible factor is HIF-2α; and the second therapeutic agent is an alkylating agent.

A fourteenth aspect may include thirteenth aspect, wherein: the first therapeutic agent is acriflavine (ACF); the third therapeutic agent is PT2385; and the alkylating agent is temozolomide (TMZ).

A fifteenth aspect may include any one of the twelfth through fourteenth aspects, wherein: the first coaxially electrospun nanofiber membrane is folded and compressed to form a first coaxially electrospun nanofiber NanoMesh; the second coaxially electrospun nanofiber membrane is folded and compressed to form a second coaxially electrospun nanofiber NanoMesh; the third coaxially electrospun nanofiber membrane is folded and compressed to form a third coaxially electrospun nanofiber NanoMesh; the first layer comprises a first disc that has been laser cut from the first coaxially electrospun nanofiber NanoMesh; the second layer comprises a second disc that has been laser cut from the second coaxially electrospun nanofiber NanoMesh; and the third layer comprises a third disc that has been laser cut from the third coaxially electrospun nanofiber NanoMesh.

A sixteenth aspect may include the fifteenth aspect, wherein: the first hypoxia-inducible factor is HIF-1α; the second hypoxia-inducible factor is HIF-2α; and the second therapeutic agent is an alkylating agent.

A seventeenth aspect may include any one of the fifteenth or sixteenth aspects, wherein: the first therapeutic agent is acriflavine (ACF); the third therapeutic agent is PT2385; and the alkylating agent is temozolomide (TMZ).

An eighteenth aspect of the present disclosure includes a method for treating cancer in a subject, the method comprising: delivering at least the first therapeutic agent and the second therapeutic agent to a target region of the subject by implanting the drug delivery device of any one of the first through seventeenth aspects at or near the target region.

A nineteenth aspect may include the eighteenth aspect, wherein the method is used to treat glioblastoma multiforme (GBM).

According to a twentieth aspect of the present disclosure, a method of manufacturing a drug delivery device comprises: preparing a first coaxially electrospun nanofiber membrane, wherein a first therapeutic agent is integrated into the first coaxially electrospun nanofiber membrane; preparing a second coaxially electrospun nanofiber membrane, wherein a second therapeutic agent is integrated into the second coaxially electrospun nanofiber membrane; folding and compressing the first coaxially electrospun nanofiber membrane to form a first coaxially electrospun nanofiber NanoMesh; folding and compressing the second coaxially electrospun nanofiber membrane to form a second coaxially electrospun nanofiber NanoMesh; laser cutting a first disc from the first coaxially electrospun nanofiber NanoMesh; laser cutting a second disc from the second coaxially electrospun nanofiber NanoMesh; and stacking the first and second discs.

A twenty-first aspect may include the twentieth aspect, further comprising pressing the first and second discs together using a heated stamp.

A twenty-second aspect may include the twenty-first aspect, wherein prior to pressing the first and second discs together, the heated stamp is heated to a sealing temperature greater than or equal to 60° C. and less than or equal to 120° C.

A twenty-third aspect may include any one of the twentieth through twenty-second aspects, further comprising: preparing a third coaxially electrospun nanofiber membrane, wherein the second therapeutic agent is also integrated into the third coaxially electrospun nanofiber membrane folding and compressing the third coaxially electrospun nanofiber membrane to form a third coaxially electrospun nanofiber NanoMesh; laser cutting a third disc from the third coaxially electrospun nanofiber NanoMesh; stacking the first, second, and third discs such that: the second disc is positioned outward from a first surface of the first disc; and the third disc is positioned outward from a second surface of the first disc, wherein the second surface of the first disc is opposite the first surface of the first disc.

A twenty-fourth aspect may include the twenty-third aspect, wherein: a first intermediate layer is positioned between the first disc and the second disc; and a second intermediate layer is positioned between the first disc and the third disc.

A twenty-fifth aspect may include the twenty-fourth aspect, further comprising pressing together, in this order, the second disc, the first intermediate layer, the first disc, the second intermediate layer, and the third disc, using a heated stamp that has been heated to a sealing temperature greater than or equal to 70° C. and less than or equal to 120° C.

According to a twenty-sixth aspect of the present disclosure, a drug delivery device comprises: a coaxially electrospun nanofiber membrane that is folded and compressed to form a coaxially electrospun nanofiber NanoMesh comprising: a first surface; a second surface opposite the first surface; and a sidewall extending around a perimeter of the coaxially electrospun nanofiber NanoMesh between the first surface and the second surface, wherein the sidewall is coated with a hydrophobic polymer coating; and a first therapeutic agent integrated into the coaxially electrospun nanofiber membrane.

A twenty-seventh aspect may include the twenty-sixth aspect, wherein the hydrophobic polymer coating comprises Teflon.

A twenty-eighth aspect may include any one of the twenty-sixth or twenty-seventh aspects, wherein the sidewall is coated twice with the hydrophobic polymer coating.

A twenty-ninth aspect may include any one of the twenty-sixth or twenty-eighth aspects, wherein the coaxially electrospun nanofiber NanoMesh comprises: the coaxially electrospun nanofiber membrane; and a homogeneously electrospun nanofiber membrane.

A thirtieth aspect may include the twenty-ninth aspect, wherein the homogeneously electrospun nanofiber membrane is hydrophobic and substantially drug-free.

A thirty-first aspect may include any one of the twenty-ninth or thirtieth aspects, wherein the coaxially electrospun nanofiber membrane and the homogeneously electrospun nanofiber membrane are arranged in an alternating fashion through a thickness of the drug delivery device.

These and other features, aspects, and advantages will become better understood with reference to the following description and the appended claims.

Additional features and advantages of the embodiments described herein will be set forth in the detailed description that follows, and in part will be readily apparent to those skilled in the art from that description or recognized by practicing the embodiments described herein, including the detailed description that follows, the claims, as well as the appended drawings.

### BRIEF DESCRIPTION OF THE DRAWINGS

FIG. 1 schematically depicts a drug delivery device of the present disclosure, according to one or more embodiments shown and described herein;

FIG. 2 is a photograph of a drug delivery device of the present disclosure, according to one or more embodiments shown and described herein;

FIG. 3 is a schematic diagram summarizing a strategy to target multiple cascades in the tumor, according to one or more embodiments shown and described herein;

FIG. 4 schematically depicts acute hypoxia at the tumor core and chronic hypoxia, normoxia at the periphery as targets for HIF-1α and HIF-2α, respectively:

FIG. 5A schematically depicts the release of a drug from a homogeneously electrospun nanofiber:

FIG. 5B schematically depicts the release of a drug from a coaxially electrospun nanofiber;

FIG. 5C schematically depicts a sustained and gradual drug release mechanism from a coaxially electrospun nanofiber membrane, according to one or more embodiments shown and described herein;

FIG. 5D schematically depicts a cross-sectional view of a multi-layer drug delivery device of the present disclosure combining a core disc having two therapeutic agents (e.g., ACF and PT2385) incorporated therein surrounded by a hydrophobic barrier layer and an outer pouch layer having a different therapeutic agent (e.g., TMZ) incorporated therein, thereby providing sequential release of multiple drugs, according to one or more embodiments shown and described herein;

FIG. 6A schematically depicts a method and apparatus for the coaxial electrospinning of core-sheath fibers, according to one or more embodiments shown and described herein;

FIG. 6B schematically depicts a magnified view showing the contents of box 6B in FIG. 6A:

FIG. 7A schematically depicts a cross-sectional view of the folding of a first electrospun nanofiber membrane (left), compression of the folded first electrospun nanofiber membrane (center) to form an first electrospun nanofiber NanoMesh, and a first NanoMesh tablet after having been laser cut from the first electrospun nanofiber NanoMesh (right), according to one or more embodiments shown and described herein:

FIG. 7B schematically depicts a magnified view showing the contents of box 7B in FIG. 7A;

FIG. 7C schematically depicts a cross-sectional view of the folding of a second electrospun nanofiber membrane (left), compression of the folded second electrospun nanofiber membrane (center) to form an second electrospun nanofiber NanoMesh, and a second NanoMesh tablet after having been laser cut from the second electrospun nanofiber NanoMesh (right), according to one or more embodiments shown and described herein;

FIG. 7D schematically depicts a cross-sectional view of the folding of a third electrospun nanofiber membrane (left), compression of the folded third electrospun nanofiber membrane (center) to form an third electrospun nanofiber NanoMesh, and a third NanoMesh tablet after having been laser cut from the third electrospun nanofiber NanoMesh (right), according to one or more embodiments shown and described herein;

FIG. 8A schematically depicts a cross-sectional view of the folding of a dual-layer electrospun nanofiber membrane (left), compression of the folded dual-layer electrospun nanofiber membrane (center) to form a dual-layer electrospun nanofiber NanoMesh, and a dual-layer tablet after having been laser cut from the dual-layer electrospun nanofiber NanoMesh (right), according to one or more embodiments shown and described herein:

FIG. 8B schematically depicts a magnified view showing the contents of box 8B in FIG. 8A;

FIG. 9A schematically depicts a cross-sectional view of an electrospun nanofiber NanoMesh wherein the sidewall of the electrospun nanofiber NanoMesh is coated with a hydrophobic polymer coating, according to one or more embodiments shown and described herein;

FIG. 9B schematically depicts a cross-sectional view a dual-layer electrospun nanofiber NanoMesh w % herein the sidewall of the dual-layer electrospun nanofiber NanoMesh is coated with a hydrophobic polymer coating, according to one or more embodiments shown and described herein;

FIG. 10 schematically depicts the stacking of layers of a drug delivery device of the present disclosure, according to one or more embodiments shown and described herein;

FIG. 11 schematically depicts the heat sealing of layers of a drug delivery device of the present disclosure, according to one or more embodiments shown and described herein;

FIG. 12A shows a scanning electron microscopy image of an electrospun nanofiber membrane containing carmustine (1,3-bis(2-chloroethyl)-1-nitrosourea (BCNU)), produced in accordance with methods of the present disclosure;

FIG. 12B shows a scanning electron microscopy image of an electrospun nanofiber membrane containing PT2385, produced in accordance with methods of the present disclosure:

FIG. 12C shows a scanning electron microscopy image of an electrospun nanofiber membrane containing acriflavine (ACF), produced in accordance with methods of the present disclosure;

FIG. 12D shows a scanning electron microscopy image of an electrospun nanofiber membrane containing temozolomide (TMZ), produced in accordance with methods of the present disclosure;

FIG. 12E shows a scanning electron microscopy image of an electrospun nanofiber membrane containing PT2385, ACF, and TMZ, produced in accordance with methods of the present disclosure:

FIG. 13A shows photographs of an electrospun nanofiber membrane (left), an electrospun nanofiber NanoMesh formed as a folded and compressed electrospun nanofiber membrane (center), and the laser cutting of NanoMesh tablets from the electrospun nanofiber NanoMesh (right), according to one or more embodiments shown and described herein:

FIG. 13B is a photograph showing tablets that have been laser cut from NanoMeshes produced in accordance with methods of the present disclosure;

FIG. 14 is a plot comparing in vitro cell viability results for various electrospun nanofiber NanoMeshes of the present disclosure, wherein time is on the x-axis and cell viability is on the y-axis;

FIG. 15A is a plot showing drug release kinetics for an electrospun nanofiber NanoMesh containing BCNU, wherein time is on the x-axis and accumulated drug release (optical density from UV-vis spectroscopy) is on the y-axis:

FIG. 15B is a plot showing drug release kinetics for an electrospun nanofiber NanoMesh containing PT2385, wherein time is on the x-axis and accumulated drug release (optical density from UV-vis spectroscopy) is on the y-axis;

FIG. 15C is a plot showing drug release kinetics for an electrospun nanofiber NanoMesh containing ACF, wherein time is on the x-axis and accumulated drug release (optical density from UV-vis spectroscopy) is on the y-axis:

FIG. 15D is a plot showing drug release kinetics for an electrospun nanofiber NanoMesh containing TMZ, wherein time is on the x-axis and accumulated drug release (optical density from UV-vis spectroscopy) is on the y-axis;

FIG. 15E is a plot showing drug release kinetics for an electrospun nanofiber NanoMesh containing PT2385, ACF, and TMZ, wherein time is on the x-axis and accumulated drug release (optical density from UV-vis spectroscopy) is on they-axis:

FIG. 15F is a plot showing drug release kinetics for a drug delivery device of the present disclosure with a TMZ membrane (outer layer) & ACF inner NanoMesh (core layers)), wherein time is on the x-axis and accumulated drug release (optical density from UV-vis spectroscopy) is on the y-axis;

FIG. 16 is a plot showing drug release kinetics for two coaxially electrospun nanofiber NanoMeshes of the present disclosure, wherein time is on the x-axis and accumulated drug release (optical density from UV-vis spectroscopy) is on the y-axis; and

FIG. 17 is a plot showing drug release kinetics for a Teflon-coated coaxially electrospun nanofiber NanoMesh and a Teflon-coated dual-layer coaxially electrospun nanofiber NanoMesh, wherein time is on the x-axis and accumulated drug release (optical density from UV-vis spectroscopy) is on the y-axis.

### DETAILED DESCRIPTION

The details of embodiments of the presently disclosed subject matter are set forth in this document. Modifications to embodiments described in this document, and other embodiments, will be evident to those of ordinary skill in the art after a study of the information provided in this document.

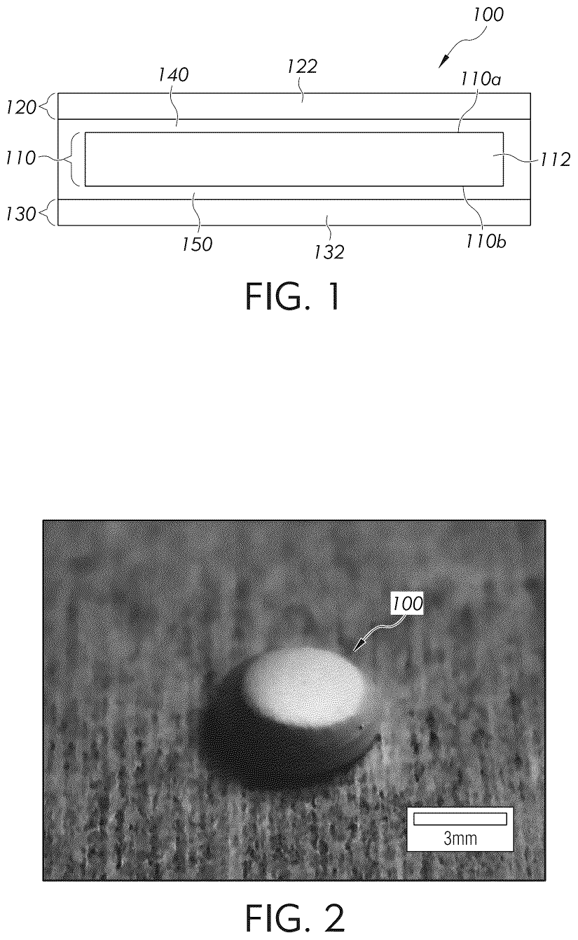

Referring now to FIG. 1, a drug delivery device 100 of the present disclosure comprises a first layer 110 comprising a first coaxially electrospun nanofiber membrane 112 and a second layer 120 comprising a second coaxially electrospun nanofiber membrane 122. The drug delivery device 100 further comprises a first therapeutic agent T1 integrated into the first coaxially electrospun nanofiber membrane 112 and a second therapeutic agent T2 integrated into the second coaxially electrospun nanofiber membrane 122, wherein the second therapeutic agent T2 is different from the first therapeutic agent T1, and wherein both the first therapeutic agent T1 and the second therapeutic agent T2 may be anti-neoplastic drugs.

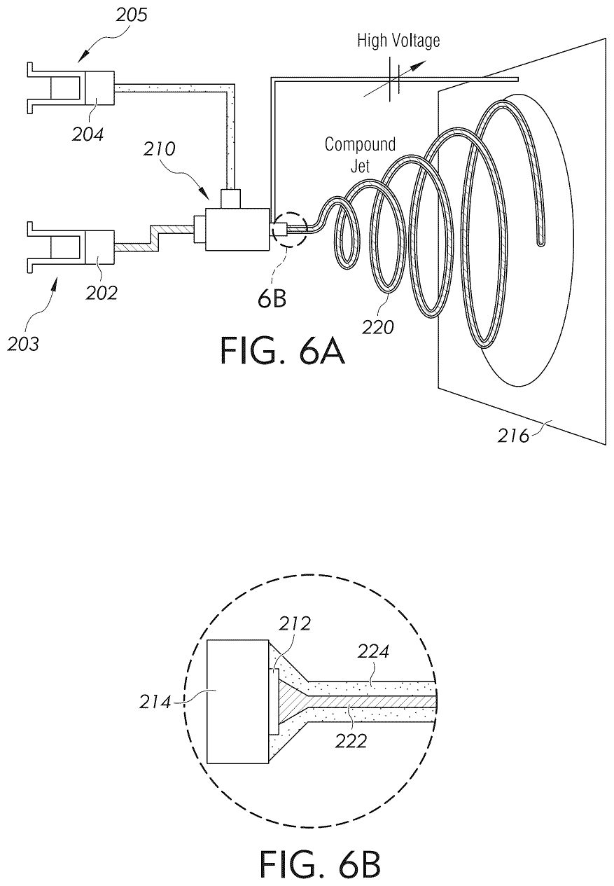

Referring now to FIGS. 6A-7C and 10, methods of manufacturing drug delivery devices of the present disclosure may comprise: preparing the first coaxially electrospun nanofiber membrane 112, wherein a first therapeutic agent T1 is integrated into the first coaxially electrospun nanofiber membrane 112; preparing a second coaxially electrospun nanofiber membrane 122, wherein a second therapeutic agent T2 is integrated into the second coaxially electrospun nanofiber membrane 122; folding and compressing the first coaxially electrospun nanofiber membrane 112 to form a first coaxially electrospun nanofiber NanoMesh 114; folding and compressing the second coaxially electrospun nanofiber membrane 122 to form a second coaxially electrospun nanofiber NanoMesh 124; laser cutting a first disc or tablet 162 from the first coaxially electrospun nanofiber NanoMesh 114; laser cutting a second disc or tablet 164 from the second coaxially electrospun nanofiber NanoMesh 124; and stacking the first and second discs or tablets 162, 164.

The drug delivery devices and methods of the present disclosure may be used to treat cancer in a subject. In particular, the drug delivery devices and methods of the present disclosure may be used to deliver the first therapeutic agent and the second therapeutic agent to a target region of the subject by implanting the drug delivery devices disclosed herein at or near the target region. The drug delivery devices and methods of the present disclosure may be used to treat glioblastoma multiforme (GBM).

While the following terms are believed to be well understood in the art, definitions are set forth to facilitate explanation of the presently disclosed subject matter. Unless defined otherwise, all technical and scientific terms used herein have the same meaning as commonly understood by one of ordinary skill in the art to which the presently disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in this specification and the appended claims, the singular forms “a,” “an,” and “the” include plural references unless the context clearly dictates otherwise.

As used herein, the term “subject” generally refers to a living being (e.g., animal or human) capable of suffering from cancer. In a specific embodiment, the subject is a mammal, such as a human, rat, mouse, monkey, horse, cow, pig, dog, cat, guinea pig, etc. In a more specific embodiment, the subject is a human subject, a rat, or a mouse. In a more specific embodiment, the subject is a human.

As used herein, the terms “treat,” “treatment,” and “treating,” refer to a method of alleviating or abrogating a disease, disorder, and/or symptoms thereof. In a specific embodiment, the disease or disorder is cancer.

As used herein, the term “therapeutic agent” means a compound utilized to image, impact, treat, combat, ameliorate, prevent, or improve an unwanted condition or disease of a patient. In embodiments, the therapeutic agent is an anti-neoplastic agent or drug. As used herein, the term “anti-neoplastic agent” or “anti-neoplastic drug” are used interchangeably and refer to a medication that is used to treat cancer. In embodiments, anti-neoplastic agents may include alkylating and alkylating-like agents, antimetabolites, antitumor antibiotics, plant alkaloids, hormonal agents, and other miscellaneous agents such as hypoxia-inducible factor (HIF) inhibitors, topoisomerase inhibitors, and others. In embodiments, the anti-neoplastic agents comprise one or more of alkylating agents, HIF-1α inhibitors, and/or HIF-2 α inhibitors.

As used herein, the term “target” refers to the cell type, tissue, or region to which enhanced delivery of the therapeutic agent is desired. For example, diseased tissue may be a target for therapy.

As used herein, the term “electrospun nanofiber membrane,” refers to a non-woven nanofiber network formed by an electrospinning process.

As used herein, the term “electrospun nanofiber NanoMesh,” sometimes abbreviated to “NanoMesh” refers to an electrospun nanofiber membrane that is folded and compressed.

Glioblastoma (GBM), the most common and highly aggressive primary central nervous system tumor. GBM is a tumor characterized by regions of pseudopalisading necrosis, which represents a hypoxic core enveloped by tumor. Several strategies have been utilized to circumvent the blood brain barrier (BBB) in order to improve the survival of patients with GBM. Among these, local delivery has played a central role over the past 30 years, achieving significant results in pre-clinical models and in humans, with the FDA approval of Gliadel® in 2003. This route allows more efficient drug delivery to the tumor bed, leading to higher therapeutic responses with minimal systemic side effects. However, despite these advances, only marginal improvement in median survival has been observed. The presence of the GBM stem cell population, as well as high tumor heterogeneity, proliferative rate, infiltrative nature and marked vascularity are the main factors limiting the efficacy of current approaches.

Studies have demonstrated that tumor hypoxia is strongly correlated with GBM stem cell phenotype, immune microenvironment, neo-angiogenesis, invasive capacity of the tumor, as well as tumor recurrence, resistance to chemo- and radio-therapy, and decreased survival rates. All of these pathways rely on the pivotal role of hypoxia-inducible factors (HIF-1α and HIF-2α) and their downstream cascades. These findings suggest that blockage of HIFs represents an opportunity to achieve a significant cytotoxic effect inside the tumor mass. Moreover, studies have found an overexpression of certain hypoxia-inducible factors (HIFs) in GBM and that overexpression has been strongly correlated with poor prognosis. Subsequent studies have further characterized these HIF-dependent pathways and identified their roles in the GBM microenvironment.

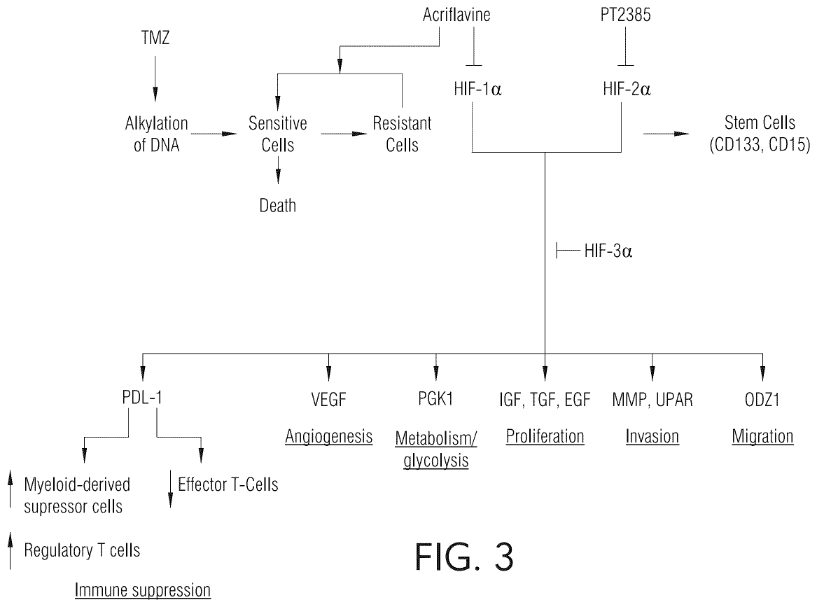

HIF-1α promotes angiogenesis and regulates metabolic pathways. HIF-2α is selectively expressed in tumor cells and plays a key role in stem cell phenotype expression. Both HIF-1α and HIF-2α have shown prognostic implications, with higher expression in tumors correlating with poor patient survival. Clinical and preclinical evidence have confirmed the efficacy of HIF-targeting in several types of neoplasms, but this strategy has so far proven of limited utility in brain tumors. Importantly, as shown in FIG. 3, HIF-1α and HIF-2α are upstream in the signaling pathway and inhibiting their signaling may result in simultaneously downregulating multiple targets. Additionally, since HIF-1α and HIF-2α are upstream targets, resistance mechanisms including compensatory pathways are limited. HIF-3α is the most recently discovered HIF family member and no specific inhibitor for it has been developed yet. While not desiring to be bound by theory, it is believed to function as a dominant negative regulator of the hypoxia response mediated by the HIF-1α and HIF-2α factors. Therefore, its expression can augment the inhibitory effect of the drugs on HIF-1α and HIF-2α. Thus, while the present disclosure is focused on drugs that inhibit hypoxia-inducible factors HIF-1α and HIF-2α, embodiments are envisioned wherein other hypoxia-inducible factors. e.g., HIF-3α, are targeted.

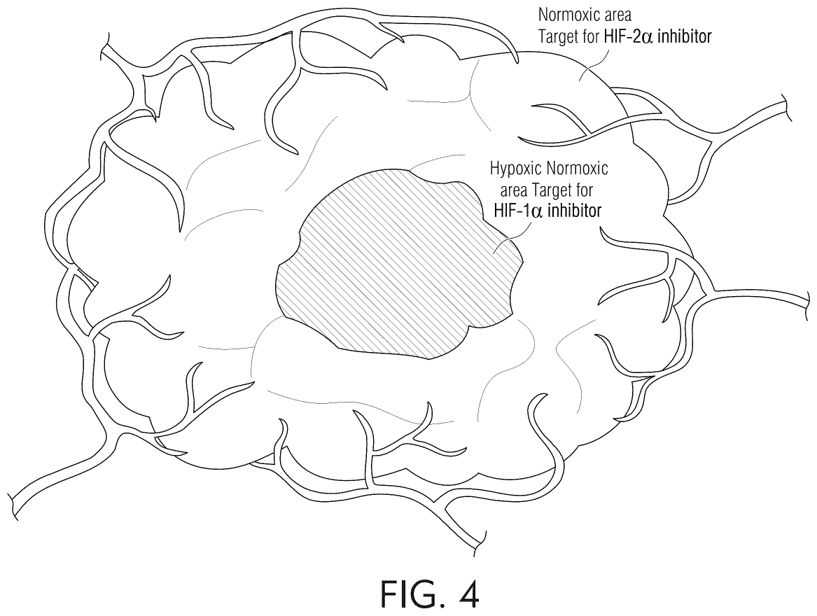

With reference to FIG. 4, HIF-1α dominates the acute response to hypoxia (in the necrotic core of the tumor), whereas HIF-2α mediates responses to chronic hypoxia and is also expressed under normoxic conditions (at the periphery of the tumor). In addition, expression of HIFs counteracts the anti-tumor immune response. Therefore, the combination of HIF-1α and HIF-2α inhibitors can maximize the opportunity for achieving a global intra-tumor cytotoxicity as well as enhancing the anti-tumor immune response. Another advantage for using HIF inhibitors is that downregulation of these HIFs, HIF-1α specifically, significantly improves the sensitivity of resistant GBM cells to alkylating agents such as temozolomide (TMZ). Therefore, combining HIF-1α and HIF-2α inhibitors with TMZ may improve the efficacy of the drug delivery devices described herein while, at the same time, reducing the number of doses required for each drug. In embodiments, the drug delivery devices of the present disclosure implement a HIF-1α- and HIF-2α-targeting strategy for GBM-treatment using intratumorally-administered acriflavine (ACF), a HIF-1α inhibitor, PT2385, a HIF-2α inhibitor, and TMZ—all delivered from electrospun polymeric membranes. It will be appreciated that various HIF inhibitors and alkylating agents are known in the art and suitable for use in the disclosed embodiments. In embodiments, alkylating agents include, but are not limited to, altretamine, bendamustine, busulfan, carmustine, chlorambucil, cyclophosphamide, dacarbazine, ifosfamide, lomustine, lurbinectedin, mechlorethamine, melphalan, procarbazine, streptozocin, temozolomide, thiotepa, trabectedin, carboplatin, cisplatin, oxaliplatin, and the like. Suitable HIF inhibitors include, but are not limited to, everolimus, camptothecin, SN-38, topotecan, irinotecan, ganetespib, acriflavine (ACF), DFF332, metformin, erlotinib, HS173, sirolimus, panobinostat, EZN2698, PX478, 2ME2, echinomycin, anthracycline, PT2385. PT2977, and the like.

The introduction of electrospun nanofiber membranes for drug delivery is aimed at providing programmed (combinations of simultaneous and sequential) multi-drug release for long-term periods to the tumor bed. Although other types of drug carriers (e.g., microspheres and gels) have been investigated, it is believed that electrospun nanofiber membranes provide unique benefits as a local drug-delivery vehicle due to their physical characteristics and dimensions. Electrospinning techniques involve extracting a liquid jet from a polymer solution by an electric field between the polymer droplet at the end of a nozzle and a collector electrode. Vigorous whipping and bending actions caused by the charge repulsion force within the liquid jet stretch the liquid jet diameter down to the nanometer range while thoroughly evaporating the solvent. The outcome of this process is a highly porous membrane that contains a non-woven nanofiber network. The physical and chemical properties of this nanofiber can be manipulated by controlling polymer concentration, additives, and solvent selection.

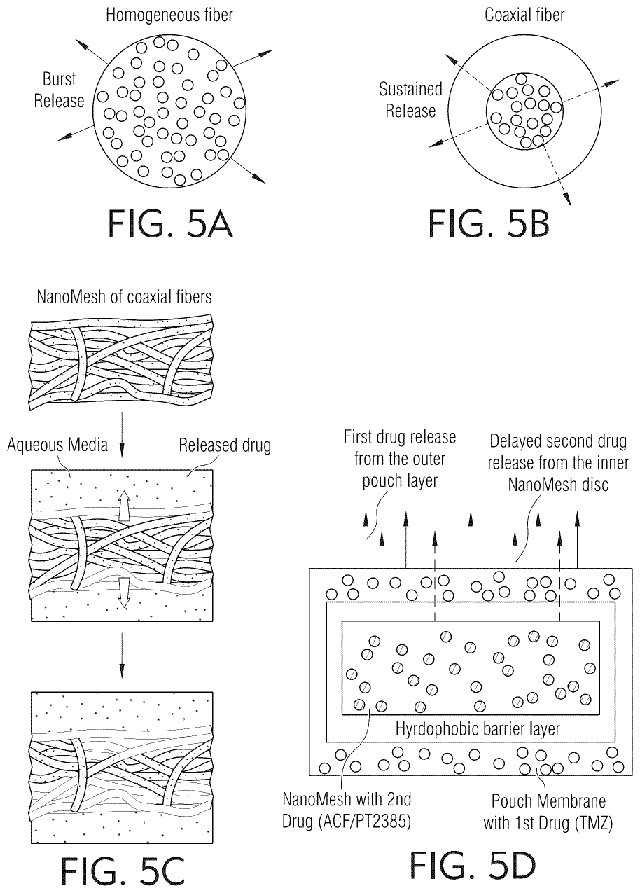

Moreover, the versatility of the electrospinning technique can be further extended by forming core-sheath fibers using coaxial nozzle electrospinning. In addition to the advantages of homogeneous fiber electrospinning, such as control of fiber morphologies and compositions, extremely high surface area and porous structure, coaxial electrospinning enables: (a) combination of polymers with two different sets of properties into one fiber; (b) encapsulation of multiple drug molecules into specified layers; and (c) control of drug release rate by optimizing fiber structure and composition. FIGS. 5A and 5B schematically illustrate how a coaxial fiber is able to achieve sustained drug release relative to a homogeneous fiber (drugs depicted as circles). In addition to sustained drug release, the sheath of the coaxially electrospun nanofibers protects active ingredients in the fiber core from hydrolytic degradation.

Coaxially electrospun nanofiber membranes may be densified and then cut into thin, e.g., 1 mm, round (discs) or square tablets (sometimes referred to herein as NanoMesh). This increases the loading capacity of incorporated drugs. Furthermore, as shown in FIG. 5C, because aqueous media gradually wets the disc from the outside due to the hydrophobicity of fiber surfaces, NanoMesh provides uniform diffusion lengths over time, leading to enhanced sustainability of drug release, with a lower initial burst release (drugs depicted as dots). While the utility of electrospun fibers for tumor research has been explored, clinically applicable core-sheath fiber membranes that outperform current standard of care treatments in terms of both consistent long-term delivery of anti-neoplastic drug and high efficacy against glioma cells in vivo, have not yet been reported.

The present disclosure is directed to producing a practical, easy-to-handle, and robust drug delivery device, sometimes referred to herein as a “NanoPouch,” that is formed as a densified multi-layer structure containing coaxially electrospun nanofiber membranes. Importantly, as shown in FIG. 5D, by selectively stacking certain membranes, programmable sequential and/or simultaneous release of one or multiple drugs can be attained. The NanoPouch shown in FIG. 5D initially releases a first anti-neoplastic agent, including but not limited to TMZ, from the outer layers (indicated by solid arrows) and then releases one or more second anti-neoplastic agents (depicted as circles) from the inner core (indicated by dashed arrows). Additionally, embodiments of the drug delivery devices of the present disclosure permits simultaneous release of multiple drugs from coaxially electrospun nanofibers. The drug delivery devices presented herein permit controlled and sustained delivery of anti-neoplastic agents, including but not limited to HIF inhibitors, such as ACF and PT2385, to the tumor bed. Moreover, the drug delivery devices of the present disclosure are capable of achieving simultaneous and sequential release of three different drugs from electrospun nanofibers. Initial data obtained by the present inventors suggests that the drug delivery devices of the present disclosure will bring significant advances to GBM local therapy.

Referring again to FIG. 1, the drug delivery devices 100 of the present disclosure may have a first layer 110 comprising a first coaxially electrospun nanofiber membrane 112 and a second layer 120 comprising a second coaxially electrospun nanofiber membrane 122. A first therapeutic agent T1 may be integrated into the first coaxially electrospun nanofiber membrane 112 and a second therapeutic agent T2 may be integrated into the second coaxially electrospun nanofiber membrane 122. In embodiments, both the first therapeutic agent T1 and the second therapeutic agent T2 are anti-neoplastic drugs, although the skilled artisan will appreciate that other agents are also suitable for inclusion in the drug delivery devices disclosed herein.

In embodiments, the first therapeutic agent T1 is selected to inhibit a first hypoxia-inducible factor. In embodiments, the first therapeutic agent T1 is a HIF-1α inhibitor. In specific embodiments, the first therapeutic agent T1 is acriflavine (ACF). However, it will be appreciated that other anti-neoplastic agents are known in the field and suitable for use as the first therapeutic agent.

In embodiments, the second therapeutic agent T2 is an alkylating chemotherapeutic agent. In a specific embodiment, the second therapeutic agent T2 is temozolomide (TMZ). In embodiments, the second therapeutic agent T2 is also integrated into the first coaxially electrospun nanofiber membrane 112. However, it will be appreciated that other anti-neoplastic agents are known in the field and suitable for use as the second therapeutic agent.

In embodiments, a third therapeutic agent T3 is integrated into the first coaxially electrospun nanofiber membrane 112 in addition to the first therapeutic agent T1. In embodiments, the third therapeutic agent T3 is selected to inhibit a second hypoxia-inducible factor different from the first hypoxia-inducible factor. In embodiments, the third therapeutic agent T3 is a HIF-2α inhibitor. In specific embodiments, the third therapeutic agent T3 is PT2385. In embodiments, the third therapeutic agent T3 is also integrated into the first coaxially electrospun nanofiber membrane 112. It will be appreciated that other anti-neoplastic agents are known in the field and suitable for use as the third therapeutic agent.

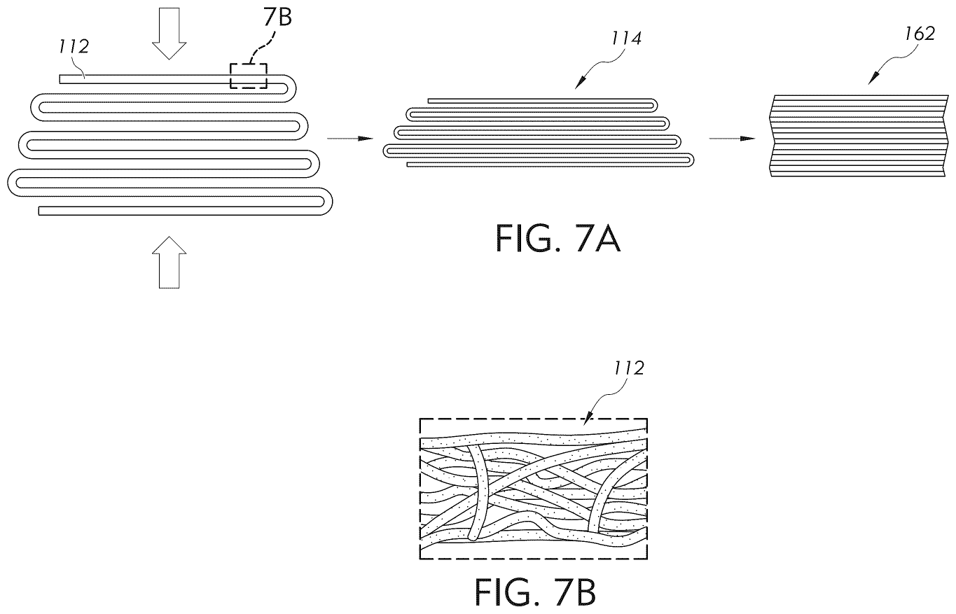

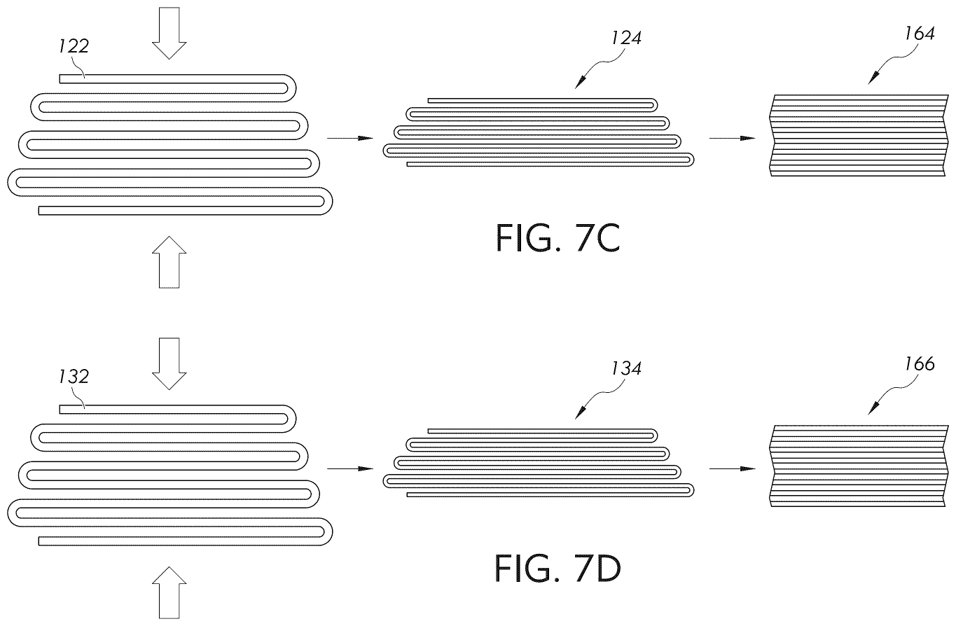

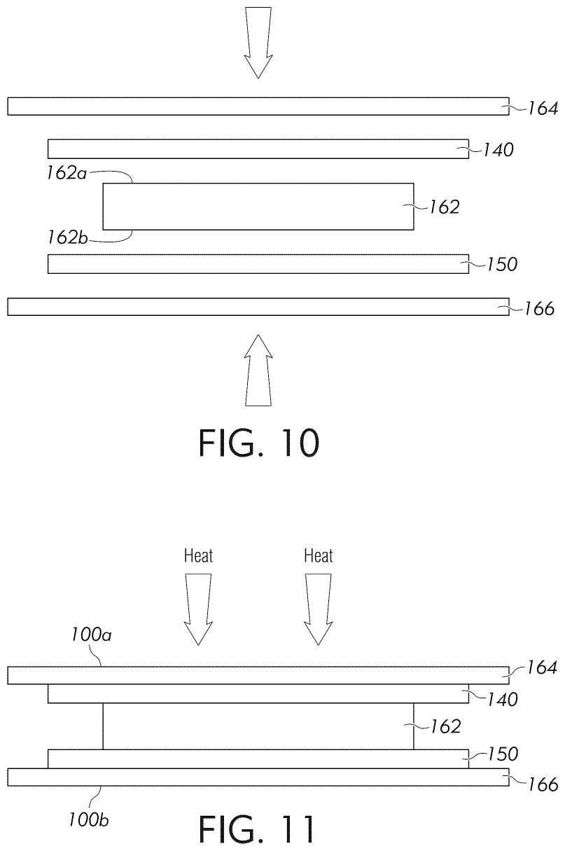

With reference to FIGS. 7A-7D and 10, the first coaxially electrospun nanofiber membrane 112 may be folded and compressed to form a first coaxially electrospun nanofiber NanoMesh 114. The first layer 110 may comprise a first disc or tablet 162 that has been laser cut from the first coaxially electrospun nanofiber NanoMesh 114. In the same manner, the second coaxially electrospun nanofiber membrane 122 may be folded and compressed to form a second coaxially electrospun nanofiber NanoMesh 124. The second layer 120 may comprise a second disc or tablet 164 that has been laser cut from the second coaxially electrospun nanofiber NanoMesh 124. The use of precision laser cutting improves the drug loading efficiency, reproducibility, and flexibility of the drug delivery devices presented herein.

As shown in FIG. 1, the drug delivery device 100 may further comprise a third layer 130 comprising a third coaxially electrospun nanofiber membrane 132. In the same manner, the third coaxially electrospun nanofiber membrane 132 may folded and compressed to form a third coaxially electrospun nanofiber NanoMesh 134. The third layer 130 may comprise a third disc or tablet 166 that has been laser cut from the third coaxially electrospun nanofiber NanoMesh 134. In embodiments, the second therapeutic agent T2 is also incorporated into the third coaxially electrospun nanofiber membrane 132. In embodiments, the second layer 120 is positioned outward from a first surface 110a of the first layer 110 and the third layer 130 is positioned outward from a second surface 110b of the first layer 110, wherein the second surface 110b of the first layer 110 is opposite the first surface 110a of the first layer 110. In this manner, a “pouch” is created wherein the first layer 110, containing the first coaxially electrospun nanofiber membrane 112, is sandwiched between the second layer 120, containing the second coaxially electrospun nanofiber membrane 122, and the third layer 130, containing the third coaxially electrospun nanofiber membrane 132.

The drug delivery devices 100 of the present disclosure may further comprise a first intermediate layer 140 interposed between the first layer 110 and the second layer 120 and a second intermediate layer 150 interposed between the first layer 110 and the third layer 130. In embodiments, one or both of the first and second intermediate layers 140, 150 is a hydrophobic barrier layer surrounding the first layer 110 of the drug delivery device 100. Moreover, in embodiments, one or both of the first and second intermediate layers 140, 150 comprises an electrospun nanofiber membrane. In embodiments, the first and second intermediate layers 140, 150 comprise homogeneously electrospun nanofiber membranes. In other embodiments, the first and second intermediate layers 140, 150 comprise coaxially electrospun nanofiber membranes. The electrospun nanofiber membranes of the first and second intermediate layers 140, 150 may be folded and compressed to form electrospun nanofiber NanoMeshes.

The coaxial nanofiber(s) forming the coaxially electrospun nanofiber membranes 112, 122, 132 may be produced in accordance with the methods described herein and may comprise a drug- and bio-compatible polymer. In embodiments, the polymers of the coaxial nanofiber(s) forming coaxially electrospun nanofiber membranes 112, 122, 132 are biodegradable. For example, the coaxial nanofiber(s) forming the coaxially electrospun nanofiber membranes 112, 122, 132 may comprise poly(s-caprolactone) (PCL), polyanhydride poly(1,3-bis(carboxyphenoxy)propane)-co-sebacic-acid (pCPP-SA), poly(lactic-co-glycolic) acid (PLGA), poly-lactic acid (PLA), polyethylene oxide (PEO), collagen, chitosan, or combinations thereof. The first and second intermediate layers 140, 150 may comprise the same polymers as the coaxially electrospun nanofiber membranes 112, 122, 132 or different polymers.

In embodiments, the coaxial electrospun nanofibers of the coaxially electrospun nanofiber membranes 112, 122, 132 have an average fiber diameter of greater than or equal to 0.1 μm and less than or equal to 3 μm, greater than or equal to 0.25 μm and less than or equal to 2.5 μm, greater than or equal to 0.3 μm and less than or equal to 2.5 μm, greater than or equal to 0.3 μm and less than or equal to 2.2 μm, or greater than or equal to 0.4 μm and less than or equal to 2.2 μm.

Similarly, the first and second intermediate layers 140, 150 may comprise electrospun nanofibers having an average fiber diameter of greater than or equal to 0.1 μm and less than or equal to 3 μm, greater than or equal to 0.25 μm and less than or equal to 2.5 μm, greater than or equal to 0.3 μm and less than or equal to 2.5 μm, greater than or equal to 0.3 μm and less than or equal to 2.2 μm, or greater than or equal to 0.4 μm and less than or equal to 2.2 μm.

With reference to FIGS. 6A-11, methods of manufacturing drug delivery devices of the present disclosure may comprise: preparing the first coaxially electrospun nanofiber membrane 112, wherein a first therapeutic agent T1 is integrated into the first coaxially electrospun nanofiber membrane 112; preparing a second coaxially electrospun nanofiber membrane 122, wherein a second therapeutic agent T2 is integrated into the second coaxially electrospun nanofiber membrane 122; folding and compressing the first coaxially electrospun nanofiber membrane 112 to form a first coaxially electrospun nanofiber NanoMesh 114; folding and compressing the second coaxially electrospun nanofiber membrane 122 to form a second coaxially electrospun nanofiber NanoMesh 124; laser cutting a first disc or tablet 162 from the first coaxially electrospun nanofiber NanoMesh 114; laser cutting a second disc or tablet 164 from the second coaxially electrospun nanofiber NanoMesh 124; and stacking the first and second discs or tablets 162, 164.

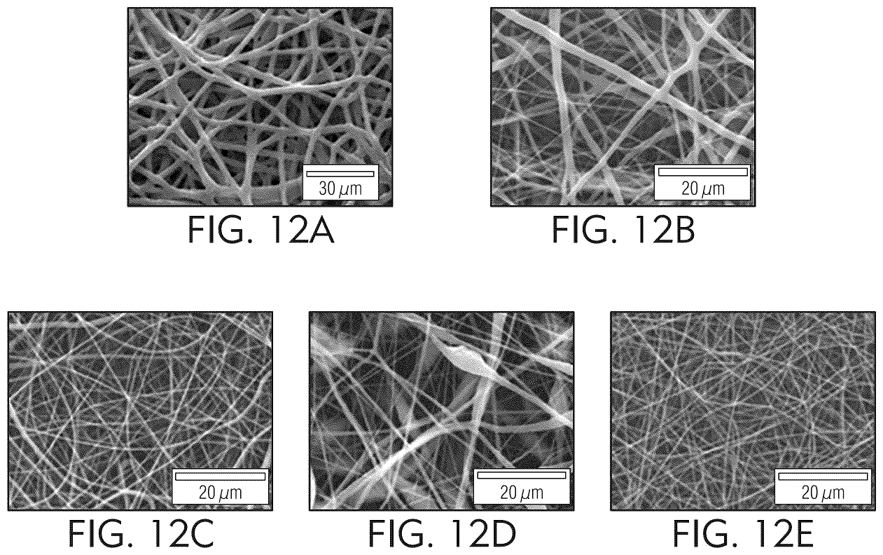

An electrospinning apparatus such as the electrospinning apparatus 200 shown in FIGS. 6A and 6B may be used for the coaxial electrospinning of nanofiber membranes such as the first coaxially electrospun nanofiber membrane 112 and the second coaxially electrospun nanofiber membrane 122. In the embodiment shown in FIGS. 6A and 6B, a first polymer solution 202 is loaded into a first syringe pump 203 and a second polymer solution 204 is loaded into a second syringe pump 205. The first and second syringe pumps 203, 205 pump the first polymer solution 202 and the second polymer solution 204 to a first nozzle 212 and a second nozzle 214, respectively. The second nozzle 214 is concentric with the first nozzle 212 and has a larger diameter such that the first nozzle 212 extends within the second nozzle 214 to form a nozzle assembly 210, as shown in FIG. 6B. The first and second polymer solutions 202, 204 are simultaneously ejected from the nozzle assembly 210 as a compound jet 220 comprising an inner core 222 and an outer sheath 224. The inner core 222 comprises the first polymer solution 202 and the outer sheath 224 comprises the second polymer solution 204. An applied electric voltage between the nozzles 212, 214 and a conductor electrode, shown as a conductor plate 216 in FIG. 6A, draws the compound jet 220 towards the conductor electrode. Vigorous whipping and bending actions caused by the charge repulsion force within the compound jet 220 stretch the compound jet 220 while thoroughly evaporating solvents from the first and second polymer solutions 202, 204. The outcome of this process is a highly porous membrane that contains a non-woven nanofiber network such as those shown in the scanning electron microscopy images of FIGS. 12A-12E. The physical and chemical properties of the nanofiber produced from electrospinning may be adjusted by controlling polymer concentration, additives, solvent selection, as well as other parameters of the electrospinning process.

In embodiments, the first polymer solution 202 comprises a drug- and bio-compatible polymer as its primary polymer component. In embodiments, the first polymer solution 202 comprises a biodegradable polymer as its primary polymer component. For example, the primary polymer component of the first polymer solution 202 comprises poly(ε-caprolactone) (PCL), polyanhydride poly(1,3-bis(carboxyphenoxy)propane)-co-sebacic-acid (pCPP-SA), poly(lactic-co-glycolic) acid (PLGA), poly-lactic acid (PLA), polyethylene oxide (PEO), collagen, chitosan, or combinations thereof. In embodiments, the PCL comprises a number average molecular weight between 20 kDa (kilodalton) and 140 kDa, between 40 kDa and 120 kDa, or between 60 kDa and 100 kDa. In embodiments, the PCL comprises a number average molecular weight of about 80 kDa such as the PCL available from MilliporeSigma (St. Louis, MO). The pCPP-SA may be obtained from Guilford Pharmaceuticals (Baltimore, MD). In embodiments, the first polymer solution 202 comprises greater than or equal to 2 wt % and less than or equal to 20 wt % of the primary polymer component, based on a total weight of the first polymer solution 202. In embodiments, the first polymer solution 202 comprises the primary polymer component in an amount greater than or equal to 4 wt % and less than or equal to 20 wt %, greater than or equal to 4 wt % and less than or equal to 18 wt %, greater than or equal to 4 wt % and less than or equal to 16 wt %, greater than or equal to 6 wt % and less than or equal to 16 wt %, greater than or equal to 6 wt % and less than or equal to 14 wt %, greater than or equal to 8 wt % and less than or equal to 14 wt %, or greater than or equal to 8 wt % and less than or equal to 12 wt %, based on a total weight of the first polymer solution 202.

The first polymer solution 202 comprises a solvent such as dichloromethane (DCM, methylene chloride), 2,2,2-trifluoroethanol (TFE), or combinations thereof. The primary polymer component of the first polymer solution 202 is dissolved in the solvent of the first polymer solution 202. In embodiments wherein the first polymer solution 202 comprises a combination of TFE and DCM solvents, the TFE:DCM weight ratio is from 10:1 to 1:10, from 5:1 to 1:5, or from 2:1 to 1:2

In embodiments, the first polymer solution 202 comprises the first therapeutic agent T1 which, as discussed above, may be configured to inhibit a first hypoxia-inducible factor. In embodiments, the first therapeutic agent T1 is a HIF-1α inhibitor. In specific embodiments, the first therapeutic agent T1 is acriflavine (ACF). The first therapeutic agent T1 may be present in the first polymer solution 202 in an amount greater than or equal to 0.1 wt % and less than or equal to 20 wt %, greater than or equal to 1.0 wt % and less than or equal to 20 wt %, greater than or equal to 3.0 wt % and less than or equal to 20 wt %, greater than or equal to 5.0 wt % and less than or equal to 20 wt %, greater than or equal to 5.0 wt % and less than or equal to 15 wt %, greater than or equal to 1.0 wt % and less than or equal to 15 wt %, greater than or equal to 1.0 wt % and less than or equal to 10 wt %, greater than or equal to 1.0 wt % and less than or equal to 5.0 wt %, or greater than or equal to 1.0 wt % and less than or equal to 4.0 wt %, based on a total weight of the first polymer solution 202.

In embodiments, the first polymer solution 202 further comprises the second therapeutic agent T2 which, as discussed above, may be an alkylating chemotherapeutic agent such as temozolomide (TMZ). The second therapeutic agent T2 may be present in the first polymer solution 202 in an amount greater than or equal to 0.1 wt % and less than or equal to 20 wt %, greater than or equal to 1.0 wt % and less than or equal to 20 wt %, greater than or equal to 3.0 wt % and less than or equal to 20 wt %, greater than or equal to 5.0 wt % and less than or equal to 20 wt %, greater than or equal to 5.0 wt % and less than or equal to 15 wt %⁰, greater than or equal to 1.0 wt % and less than or equal to 15 wt %, greater than or equal to 1.0 wt % and less than or equal to 10 wt %, greater than or equal to 1.0 wt % and less than or equal to 5.0 wt %, greater than or equal to 2.0 wt % and less than or equal to 5.0 wt %, or greater than or equal to 1.0 w % and less than or equal to 4.0 wt %, based on a total weight of the first polymer solution 202.

In embodiments, the first polymer solution 202 further comprises the third therapeutic agent T3 which, as discussed above, may be configured to inhibit a second hypoxia-inducible factor different from the first hypoxia-inducible factor. In embodiments, the third therapeutic agent T3 is a HIF-2α inhibitor. In specific embodiments, the third therapeutic agent T3 is PT2385. The third therapeutic agent T3 may be present in the first polymer solution 202 in an amount greater than or equal to 0.1 wt % and less than or equal to 20 wt %, greater than or equal to 1.0 wt % and less than or equal to 20 wt %, greater than or equal to 3.0 wt % and less than or equal to 20 wt %, greater than or equal to 5.0 wt % and less than or equal to 20 wt %, greater than or equal to 5.0 wt % and less than or equal to 15 wt %, greater than or equal to 1.0 wt % and less than or equal to 15 wt %, greater than or equal to 1.0 wt % and less than or equal to 10 wt %, greater than or equal to 1.0 wt % and less than or equal to 5.0 wt %, greater than or equal to 2.0 wt % and less than or equal to 5.0 wt %, or greater than or equal to 1.0 wt % and less than or equal to 4.0 wt %, based on a total weight of the first polymer solution 202.

In embodiments, the flow rate of the first polymer solution 202 through the first nozzle 212 is greater than or equal to 0.1 mL/hr (milliliters per hour) and less than or equal to 3.0 mL/hr, greater than or equal to 0.3 mL/hr (milliliters per hour) and less than or equal to 2.5 mL/hr, or greater than or equal to 0.5 mL/hr (milliliters per hour) and less than or equal to 2.0 mL/hr.

In embodiments, the second polymer solution 204 comprises a biocompatible and hydrophobic polymer, such as the PCL discussed above, as its primary polymer component. The second polymer solution 204 may comprise greater than or equal to 2 wt % and less than or equal to 20 wt % of the primary polymer component, based on a total weight of the second polymer solution 204. In embodiments, the second polymer solution 204 comprises the primary polymer component in an amount greater than or equal to 4 wt % and less than or equal to 20 wt %, greater than or equal to 4 wt % and less than or equal to 18 wt %, greater than or equal to 4 wt % and less than or equal to 16 wt %, greater than or equal to 6 wt % and less than or equal to 16 wt %, greater than or equal to 6 wt % and less than or equal to 14 wt %, greater than or equal to 8 wt % and less than or equal to 14 wt %, or greater than or equal to 8 wt % and less than or equal to 12 wt %, based on a total weight of the second polymer solution 204.

The second polymer solution 204 comprises a solvent such as dichloromethane (DCM, methylene chloride), 2,2,2-trifluoroethanol (TFE, 99.8% purity), or combinations thereof. The primary polymer component of the second polymer solution 204 is dissolved in the solvent of the second polymer solution 204. In embodiments wherein the second polymer solution comprises a combination of TFE and DCM solvents, the TFE:DCM weight ratio may be from 10:1 to 1:10, from 5:1 to 1:5, or from 2:1 to 1:2.

In embodiments, the second polymer solution 204 further comprises the second therapeutic agent T2 which, as discussed above, may be an alkylating chemotherapeutic agent such as temozolomide (TMZ). The second therapeutic agent T2 may be present in the second polymer solution 204 in an amount greater than or equal to 0.1 wt % and less than or equal to 30 wt %, greater than or equal to 0.1 wt % and less than or equal to 20 wt %, greater than or equal to 1.0 wt % and less than or equal to 20 wt %, greater than or equal to 3.0 wt % and less than or equal to 20 wt %, greater than or equal to 5.0 wt % and less than or equal to 20 wt %, greater than or equal to 5.0 wt % and less than or equal to 15 wt %, greater than or equal to 1.0 wt % and less than or equal to 15 wt %, greater than or equal to 1.0 wt % and less than or equal to 10 wt %, greater than or equal to 1.0 wt % and less than or equal to 6.0 wt %, greater than or equal to 1.0 wt % and less than or equal to 5.0 wt %, or greater than or equal to 1.0 wt % and less than or equal to 4.0 wt %, based on a total weight of the second polymer solution 204.

In embodiments, the flow rate of the second polymer solution 204 through the second nozzle 214 is greater than or equal to 0.1 mL/hr (milliliters per hour) and less than or equal to 2.0 mL/hr, greater than or equal to 0.3 mL/hr (milliliters per hour) and less than or equal to 1.5 mL/hr, or greater than or equal to 0.5 mL/hr (milliliters per hour) and less than or equal to 1.2 mL/hr.

The physical gap (i.e., the separation distance) between the outlet of the first and second nozzles 212, 214 and the conductor electrode, i.e., the collecting substrate for the electrospun nanofiber mesh, may be greater than or equal to 5 cm (centimeters) and less than or equal to 50 cm, greater than or equal to 10 cm and less than or equal to 40 cm, greater than or equal to 15 cm and less than or equal to 40 cm, or greater than or equal to 15 cm and less than or equal to 30 cm. The electric voltage between the nozzles 212, 214 and the conductor electrode may be greater than or equal to 5 kV and less than or equal to 40 kV, greater than or equal to 10 kV and less than or equal to 40 kV, greater than or equal to 10 kV and less than or equal to 30 kV, or greater than or equal to 10 kV and less than or equal to 23 kV.

The physical gap and the electric voltage between outlet of the nozzles 212, 214 and the conductor electrode, along with the respective parameters and the flow rates of the first and second polymer solutions 202, 204, may be adjusted to achieve desired characteristics of the coaxially electrospun nanofiber. Moreover, the physical gap and the electric voltage between outlet of the nozzles 212, 214 and the conductor electrode, along with the respective parameters and the flow rates of the first and second polymer solutions 202, 204, may be adjusted to achieve desired characteristics of the first and second nanofiber meshes produced from the electrospinning process. In embodiments, the parameters of the electrospinning process are adjusted such that the electrospun nanofiber comprises an average fiber diameter of greater than or equal to 0.1 μm and less than or equal to 3 μm, greater than or equal to 0.25 μm and less than or equal to 2.5 μm, greater than or equal to 0.3 μm and less than or equal to 2.5 μm, greater than or equal to 0.3 μm and less than or equal to 2.2 μm, or greater than or equal to 0.4 μm and less than or equal to 2.2 μm.

While not shown in FIG. 6A or 6B, triaxial electrospinning may also be utilized to further control the drug release characteristics from the nanofiber. With the use of an additional concentric nozzle, an intermediate sheath layer is formed within the compound jet 220 radially inwards from the outer sheath 224, but radially outwards from the inner core 222. Adjusting various parameters such as polymer solution composition, polymer concentration, flow rate ratio, etc. enables control of the molecular (i.e. drug) release rate from a short-term (few hours) to long-term (months) time period. Moreover, the combination of short- and long-term release profiles has been realized using triaxial electrospun fibers by incorporating two functional molecules into different layers. In embodiments wherein the intermediate sheath layer is hydrophobic, sustained release of molecules encapsulated in the inner core 222 may be attributable to the barrier effect of the hydrophobic intermediate layer. Moreover, “on-demand” triggered release of encapsulated materials may be achieved using external stimuli. When triggered by a targeted stimulus, the outer sheath 224 is depolymerized and exposes the inner core 222.

Homogeneous fibers formed by conventional single nozzle electrospinning have drug release profiles that are strongly affected by high drug solubility (and rapid release) in aqueous media. However, in core-sheath fibers, the effect of drug solubility can be minimized since the drugs encapsulated in the inner core 222 are shielded by the hydrophobic sheath layer 224. This benefit is significant when targeted to long-term release with hydrophilic drugs. Core-sheath fibers also provide higher drug encapsulation efficiency (˜100%) and better protection and stability from harsh environments. Utilizing coaxial electrospinning to form the nanofiber meshes of the drug delivery devices of the present disclosure allows for long-term release kinetics independent of the nature of the encapsulated therapeutic agent(s).

The electrospun nanofiber membranes of the drug delivery devices of the present disclosure, e.g., the first coaxially electrospun nanofiber membrane 112, the second coaxially electrospun nanofiber membrane 122, and the third coaxially electrospun nanofiber membrane 132, may be produced in accordance with the electrospinning apparatus and methods described above. Moreover, as mentioned above, methods of manufacturing the drug delivery devices of the present disclosure may include folding and compressing the coaxially electrospun nanofiber membranes to form coaxially electrospun nanofiber NanoMesh, e.g., the first coaxially electrospun nanofiber NanoMesh 114, the second coaxially electrospun nanofiber NanoMesh 124, and the third coaxially electrospun nanofiber NanoMesh 134. Folding the coaxially electrospun nanofiber membranes may comprise folding the membranes at least two times, at least three times, at least four times, at least five times, at least six times, at least seven times, at least eight times, at least nine times, or at least ten times. In embodiments, the coaxially electrospun nanofiber membranes are folded in a first direction 1-3 times before being folded in a different direction, i.e., a direction perpendicular to the first direction, 1-3 times, before being folded again in the first direction 1-3 times.

With reference to FIGS. 7A-7D, the folded coaxially electrospun nanofiber membranes may then be compressed, e.g., by two metal plates (not shown in FIGS. 7A and 7B), to form the coaxially electrospun nanofiber NanoMesh. After compression, the resulting coaxially electrospun nanofiber NanoMesh may have a thickness that is less than or equal to 90%, less than or equal to 80%, less than or equal to 70%, less than or equal to 60%, less than or equal to 50%, or less than or equal to 40% of the thickness of the folded coaxially electrospun nanofiber membranes prior to the compression. Folding and compressing the coaxially electrospun nanofiber membranes to form coaxially electrospun nanofiber NanoMesh increases the drug concentration (i.e., drug weight per volume) by reducing the porosity of the non-woven nanofiber network formed by the electrospinning process (typically having a porosity of around 80%).

With continued reference to FIGS. 7A-7D, a first disc or tablet 162 may be laser cut from the first coaxially electrospun nanofiber NanoMesh 114. Similarly, a second disc or tablet 164 and third disc or tablet 166 may be cut from the second coaxially electrospun nanofiber NanoMesh 124 and the third coaxially electrospun nanofiber NanoMesh 134, respectively. The methods of manufacturing the drug delivery devices of the present disclosure may comprise stacking the first disc or tablet 162 and the second disc or tablet 164, or in embodiments including the third disc or tablet 166, stacking the first disc or tablet 162, the second disc or tablet 164, and the third disc or tablet 166. With reference to FIG. 10, in embodiments of the method wherein the first disc or tablet 162, the second disc or tablet 164, and the third disc or tablet 166 are stacked, the second disc or tablet 164 may be positioned outward from a first surface 162a of the first disc or tablet 162 and the third disc or tablet 166 may be positioned outward from a second surface 162b of the first disc or tablet 162, wherein the second surface 162b of the first disc or tablet 162 is opposite the first surface 162a of the first disc or tablet 162.

Methods of manufacturing the drug delivery devices of the present disclosure may further comprise positioning the first intermediate layer 140 between the first disc or tablet 162 and the second disc or tablet 164 and positioning a second intermediate layer 150 between the first disc or tablet 162 and the third disc or tablet 166, as shown in FIG. 10. As discussed above, the first and second intermediate layers 140, 150 may comprise electrospun nanofiber membranes. Moreover, the electrospun nanofiber membranes of the first and second layers 140, 150 may be folded and compressed to form electrospun nanofiber NanoMeshes. In embodiments, the first and second intermediate layers 140, 150 are produced using a homogeneous electrospinning process using a drug-free polymer solution as the source material. The drug-free polymer solution used to form the electrospun nanofiber membranes of the first and second intermediate layers 140, 150 may comprise a biocompatible and hydrophobic polymer, such as the PCL discussed above, as its primary polymer component. When placed in a wet environment, the second therapeutic agent T2, e.g., TMZ, integrated into the second and third discs or tablets 164, 166 is initially released from this NanoPouch structure. The first therapeutic agent T1 and the third therapeutic agent T3 (if present), e.g., ACF and PT2385, are integrated into the first disc or tablet 162 are then released after penetration of water through hydrophobic intermediate layers 140, 150. Therefore, the physical and surface properties of the intermediate layers 140, 150 significantly affect the timing of the drug release from the core of the NanoPouch, i.e., the first disc or tablet 162. Release sequences of integrated drugs may be controlled by loading location and may be optimized by evaluating their timing/synergistic effects on GBM tumors. The versatile coaxial fiber-based drug delivery devices described herein enables novel local chemotherapy approaches that are not provided by other platforms, such as solids, hydrogels, microparticles and nanoparticles.

In embodiments, the drug-free polymer solution comprises greater than or equal to 2 wt % and less than or equal to 30 wt % of the primary polymer component, based on a total weight of the drug-free polymer solution. In embodiments, the drug-free polymer solution comprises the primary polymer component in an amount greater than or equal to 4 wt % and less than or equal to 30 wt %, greater than or equal to 4 wt % and less than or equal to 25 wt %, greater than or equal to 4 wt % and less than or equal to 20 wt %, greater than or equal to 4 wt % and less than or equal to 18 wt %, greater than or equal to 4 wt % and less than or equal to 16 wt %, greater than or equal to 6 wt % and less than or equal to 16 wt %, greater than or equal to 6 wt % and less than or equal to 14 wt %, greater than or equal to 8 wt % and less than or equal to 14 wt %, or greater than or equal to 8 wt % and less than or equal to 12 wt %, based on a total weight of the drug-free polymer solution.

The drug-free polymer solution comprises a solvent such as dichloromethane (DCM, methylene chloride), 2,2,2-trifluoroethanol (TFE, 99.8% purity), or combinations thereof. The primary polymer component of the drug-free polymer solution is dissolved in the solvent of the drug-free polymer solution. In embodiments wherein the second polymer solution comprises a combination of TFE and DCM solvents, the TFE:DCM weight ratio may be from 10:1 to 1:10, from 5:1 to 1:5, or from 2:1 to 1:2.

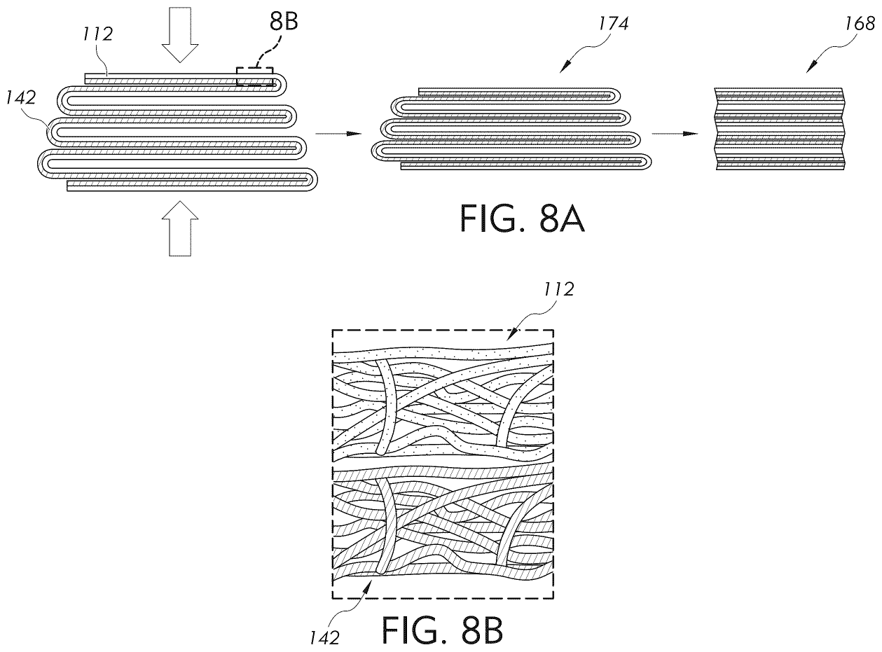

In embodiments, the drug delivery devices or the present disclosure comprise a dual-layer electrospun nanofiber NanoMesh 174 containing at least two electrospun nanofiber membranes that have been stacked, folded, and compressed (see FIGS. 8A, 8B). It should be noted that “dual-layer” indicates the presence of two different types of electrospun nanofiber membranes as opposed to a quantity of layers. By stacking multiple electrospun nanofiber membranes prior to folding, a multi-layered NanoMesh may be produced wherein electrospun nanofiber membranes having different drugs or no drugs at all alternate through the thickness of the NanoMesh. For example, with reference to FIGS. 8A and 8B, the first coaxially electrospun nanofiber membrane 112 may be stacked on a drug-free electrospun nanofiber membrane 142 (e.g., produced in the manner described above for the first and second intermediate layers 140, 150), folded and compressed to form the dual-layer electrospun nanofiber NanoMesh 174 (e.g., a pattern comprising drug-free, drug-free, drug-loaded, drug loaded, drug-free, drug-free, drug-loaded, drug-loaded, and so on). In embodiments, the drug-free electrospun nanofiber membrane 142 is substantially drug-free and hydrophobic.

Moreover, as discussed for other embodiments, a dual-layer disc 168 may then be laser cut from the dual-layer electrospun nanofiber NanoMesh 174. Without wishing to be bound by theory, a drug delivery device having alternating layers of drug-included membranes and drug-free membranes is believed to be capable of particular time release profiles that would not be attainable without these alternating layers. Moreover, without wishing to be bound by theory, the dual-layer electrospun nanofiber NanoMesh 174 is believed to be particularly advantageous when attempting to incorporate two or more drugs with different electrospinning solution solubilities (e.g., due to differing degrees of hydrophobicity) into a layer of the drug delivery devices incorporated herein. In this manner, two or more drugs may be incorporated into a layer of the drug delivery device within separate nanofiber membranes which, when stacked, folded, and compressed, result in a compact structure containing multiple drugs separated within their own respective nanofiber membranes.

With reference now to FIG. 11, the methods of the present disclosure may further comprise pressing the discs and intermediate layers (if present) together using a heated stamp that directs heat through the thickness of the drug delivery device to adhere the discs and intermediate layers (if present) together. In embodiments, the heated stamp comprises a single heated element (e.g., a heated metal plate, optionally shaped to fit the drug delivery device to be pressed) that, for example, is pressed down on an upper surface 100a drug delivery device. In other embodiments, the heated stamp comprises an upper heated element and a lower heated element (e.g., two heated metal plates, optionally shaped to fit the drug delivery device to be pressed) that are pressed against the upper surface 100a and a lower surface 100b of the drug delivery device, respectively.

In embodiments, the second disc or tablet 164, the first intermediate layer 140, the first disc or tablet 162, the second intermediate layer 150, and the third disc or tablet 166, in this order, are pressed together using a heated stamp (not shown) that has been heated to a sealing temperature greater than or equal to 60° C. and less than or equal to 120° C., greater than or equal to 75° C. and less than or equal to 115° C., greater than or equal to 80° C. and less than or equal to 110° C., greater than or equal to 85° C. and less than or equal to 105° C., greater or greater than or equal to 90° C. and less than or equal to 100° C.

In embodiments, pressing together the second disc or tablet 164, the first intermediate layer 140, the first disc or tablet 162, the second intermediate layer 150, and the third disc or tablet 166 using the heated stamp pinches and seals perimeters edges of the second disc or tablet 164, the first intermediate layer 140, the second intermediate layer 150, and the third disc or tablet 166 around the perimeter edge of the first disc or tablet 162. In this manner, the first disc or tablet 162, containing one or more therapeutic agents T1, T2, and T3 of the present disclosure, is sealed from the outer environment by at least the first and second intermediate layers 140, 150 (see FIG. 1).

In order to promote effective sealing around the perimeter edge of the first disc or tablet 162, the first disc or tablet 162 may have a characteristic in-plane dimension that is smaller than the corresponding characteristic in-plane dimension of the second disc or tablet 164, the first intermediate layer 140, the second intermediate layer 150, and the third disc or tablet 166. For example, in the case of circularly shaped discs, the characteristic in-plane dimension is the in-plane diameter of the circularly shaped disc, and the in-plane diameter of the first disc or tablet 162 is smaller than the in-plane diameters of the second disc or tablet 164, the first intermediate layer 140, the second intermediate layer 150, and the third disc or tablet 166. For square-shaped tablets, the characteristic in-plane dimension is a side length of the square. For irregular shaped discs, the characteristic in-plane dimension is the largest in-plane dimension. Moreover, as shown in FIG. 10, while being larger than the characteristic in-plane dimension of the first disc or tablet 162, the characteristic in-plane dimensions of the first and second intermediate layers 140, 150 may be smaller than the corresponding characteristic in-plane dimensions of the second disc or tablet 164 and the third disc or tablet 166.

In embodiments, the edges of the drug delivery devices presented herein are provided with a hydrophobic polymer coating to avoid high initial release of drugs integrated into the drug delivery devices. For example, when a disc or tablet is laser cut from a coaxially electrospun nanofiber NanoMesh, core regions of the coaxial nanofibers may become exposed around the perimeter edge of the disc or tablet. With these core regions of the coaxial nanofibers exposed, when the drug delivery device is placed in a wet environment, e.g., a tumor bed following surgical resection of the tumor, a high and potentially undesirable initial drug release may result. The present inventors have found that by coating the edges the perimeter edge of electrospun nanofiber NanoMeshes with a hydrophobic polymer coating, this high initial drug release can be avoided. The hydrophobic polymer coating may be particular advantageous when hydroscopic drugs are integrated into the drug delivery device.

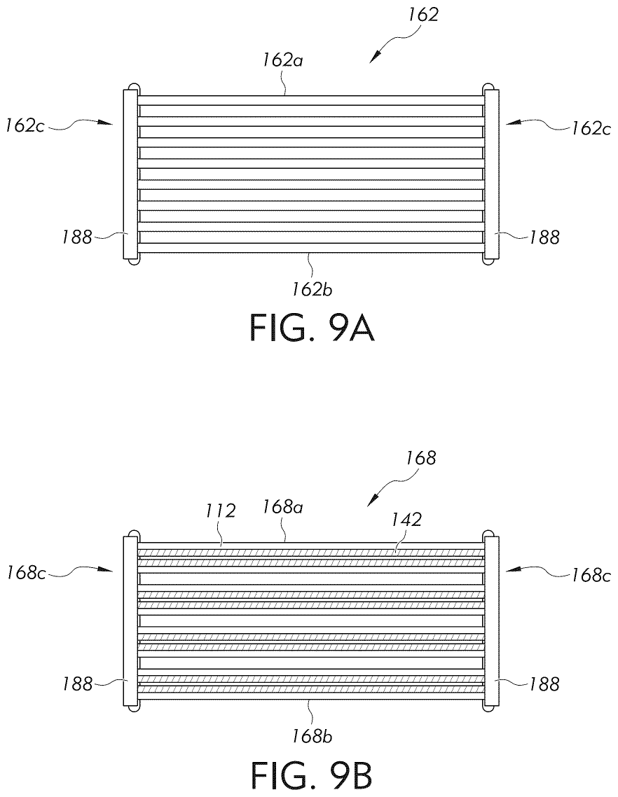

With reference now to FIG. 9A, drug delivery devices of the present disclosure may comprise a first disc or tablet 162 (e.g., laser cut from the coaxially electrospun nanofiber NanoMesh 114) having a first surface 162a, a second surface 162b opposite the first surface 162a, and a sidewall 162c extending around a perimeter of the first disc or tablet 162 between the first surface 162a and the second surface 162b, wherein the sidewall 162c is coated with a hydrophobic polymer coating 188. In embodiments, the hydrophobic polymer coating 188 is biodegradable. In embodiments, the hydrophobic polymer coating 188 comprises Teflon, e.g., Teflon AF (amorphous fluoropolymer). The sidewall 162c may be coated two or more times with the hydrophobic polymer coating 188.

In embodiments, the hydrophobic polymer coating 188 is applied to the sidewall 162c by applying a coating solution to the sidewall 162c with an appropriate applicator, e.g., a cotton tip. In embodiments, the coating solution comprises the hydrophobic polymer in an amount greater than or equal to 0.1 wt % and less than or equal to 20 wt %, greater than or equal to 0.1 wt % and less than or equal to 15 wt %, greater than or equal to 0.1 wt % and less than or equal to 5 wt %, greater than or equal to 0.5 wt % and less than or equal to 5 wt %, or greater than or equal to 0.5 wt % and less than or equal to 2 wt %, based on a total weight of the coating solution. In embodiments, the coating solution comprises the hydrophobic polymer in an amount less than 20 wt % based on the total weight of the coating solution. In embodiments, the coating solution comprises a non-polar organic solvent such as chloroform, dichloromethane, or combinations thereof. In embodiments, the coating solution comprises a fluorocarbon-based solvent such as Fluorinet™ FC-40 or FC-75.

With reference now to FIG. 9B, drug delivery devices of the present disclosure may comprise the dual-layer disc 168 (e.g., laser cut from the dual-layer electrospun nanofiber NanoMesh 174) having the first coaxially electrospun nanofiber membrane 112 and the drug-free electrospun nanofiber membrane 142, wherein a sidewall 168c of the dual-layer disc 168 is coated with the hydrophobic polymer coating 188 as described above with respect to the first disc or tablet 162. In this manner, the coaxially electrospun nanofiber membrane 112 and the drug-free electrospun nanofiber membrane 142 may be arranged in an alternating fashion through the thickness of the dual-layer disc 168, and the hydrophobic polymer coating 188 coated on the sidewall 168c prevents high initial drug release, as described above. Without wishing to be bound by theory, the combination of these features permits favorable release kinetics for the therapeutic agent(s) incorporated into the drug delivery devices. In particular, as the hydrophobic polymer coating 188 prevents drug release through the sidewall 168c of the dual-layer disc 168, and moreover because the dual-layer disc 168 is wetted from the outer layers to the inner layers (i.e., from the top to bottom surfaces), the presence of the alternating hydrophobic drug-free membranes delays release from the drug-loaded membranes of the drug delivery device. This delayed release is advantageous because high initial drug release may cause negative side effects for the subject undergoing treatment with the use of the drug delivery device.

The present disclosure is also directed to drug delivery devices and methods for treating cancer in a subject and, in particular, for the treatment of glioblastoma multiforme (GBM). Following surgical resection of a tumor, the method of treating cancer in a subject may involve implanting a drug delivery device presented herein at or near a target area corresponding to the location in which the tumor was surgically excised. In this manner, the method of treating cancer may be used to deliver at least the first therapeutic agent T1 and the second therapeutic agent T2 to the target region of the subject.

As will be understood by those of skill in this art, the specific dose level for any particular subject will depend on a variety of factors, including the activity of the agent employed; the age, body weight, general health, and sex of the individual being treated; the time and route of administration: the rate of excretion; and the like.

### EXAMPLES

The following examples are given by way of illustration are not intended to limit the scope of the disclosure.

### Example 1. Preparation of Electrospun Nanofiber Membranes

Table 1 below provides various polymer, drug, and solvent combinations used to produce the electrospun nanofiber membranes of the present disclosure.

TABLE 1
Core and Sheath Solution Parameters used for
Producing Electrospun Nanofiber Membranes

| | | | Core & |
|---|---|---|---|
| | | | sheath |
| Sample | | | flow rates |
| ID | Core solution | Sheath solution | (mL/hr) |
| BCNU | PCPP-SA 10% + 2% | PCL 10% in TFE:DCM | 0.6 & 0.8 |
| | BCNU in DCM | (6:4 wt. ratio) | |
| ACF | PCL 8.83% + ACF 1.82% | PCL 10% in TFE:DCM | 0.5 & 0.5 |
| 10% | in TFE:DCM (6:4) | (8:2) | |
| TMZ | PCL 5.72% + TMZ 2.6% | PCL 6.43% in TFE:DCM | 1.0 & 1.0 |
| 20% | in TFE:DCM (3:4) | (8:2) | |
| PT2385 | PCL 6.23% + PT2385 | PCL 7% in TFE:DCM | 1.0 & 1.0 |
| 20% | 2.83% in TFE:DCM (6:4) | (8:2 wt. ratio) | |
| 3 Drugs | PCL 4.55% + PT2385 | PCL 7% in TFE:DCM | 0.5 & 0.5 |
| | 3.64% + ACF 1.82% + | (8:2) | |
| | TMZ 3.64% in | | |
| | TFE:DCM (6:4) | | |

| | | |
|---|---|---|
| Control | PCL 7% in TFE:DCM (7:3) | 2.0 |

The core/sheath solutions were prepared by dissolving selected host polymer (primary polymer component) and drugs in the corresponding solvent(s). Due to the non-polarity and high volatility of DCM. TFE is added to improve the electrospinning process by lowering vapor pressure while maintaining good polymer solubility. All solutions are homogenized overnight at 20 RPM (revolutions per minute). Each solution is then loaded into a syringe and uniformly fed into the corresponding coaxial nozzle outlet by a corresponding syringe pump. Core and sheath solutions were fed at 1:1 flow ratio, except for the BCNU (bis-chloroethyl-nitrosourea) membrane, wherein the core and sheath solutions were fed at 0.6 and 0.8 mL/hr, respectively. An external voltage (˜15 to 22 kV) was applied across a roughly 20 cm gap between the coaxial nozzle and the collecting substrate. For control samples, the fiber was formed using single nozzle electrospinning to form homogeneously electrospun nanofibers.

Scanning electron microscopy images of the electrospun nanofiber meshes formed from the combinations shown in Table 1 are shown in FIGS. 12A-12E ((FIG. 12A) BCNU; (FIG. 12B) PT2385 20%; (FIG. 12C) ACF 10%; (FIG. 12D) TMZ 20%; (FIG. 12E) 3 Drugs (PT2385/ACF/TMZ)). Uniform fiber formation is obtained for no-drug control, BCNU, PT2385. ACF, “3 Drugs” NanoMeshes with average fiber diameters of 0.9 μm+0.4 μm, 2.2 μm+0.2 μm, 0.7 μm±0.5 μm, 0.41 μm±0.07 μm, and 0.44 μm±0.16 μm, respectively. TMZ-only incorporated fibers presented the bead-in-fiber morphology with the fiber diameter of 0.49 μm±0.16 μm and the bead diameters of 4.48 μm±1.08 μm.

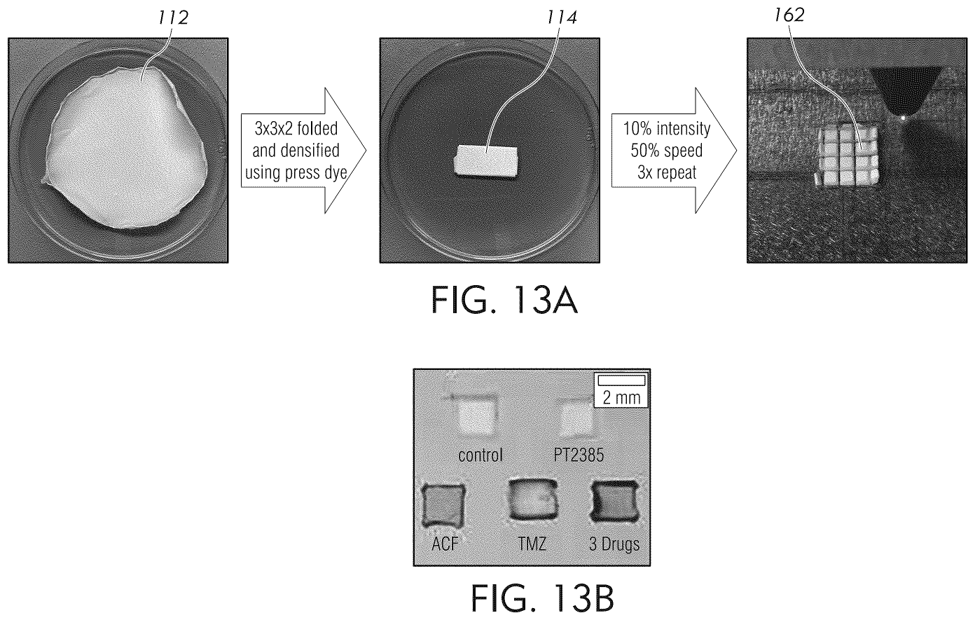

The electrospun nanofiber membranes were then removed from the collecting substrate of the electrospinning apparatus. An exemplary isolated nanofiber membrane is shown in FIG. 13A (left). The electrospun nanofiber membrane is then subjected to a 3×3×2 folding process and densified using a press dye. An exemplary resulting electrospun nanofiber NanoMesh is shown in FIG. 13A (center). Following folding and compression, NanoMesh tablets are then laser cut from the electrospun nanofiber NanoMesh, as shown in FIG. 13A (right). FIG. 13B is a photograph showing NanoMesh tablets that have been laser cut from NanoMeshes formed from electrospun nanofiber membranes produced in accordance with methods of the present disclosure

The release kinetics from each of the electrospun nanofiber NanoMesh produced according to the above procedure were then measured in accordance with the following measurement technique.

Drug release kinetic measurement: To characterize the drug release kinetics from NanoMesh samples, optical spectra of drugs were analyzed using the Thermo Scientific NanoDrop One UV-vis spectrometer. The prepared NanoMesh discs were immersed into 1.2 mL of ultrapure water contained in 1.5 mL microcentrifuge tubes. Then, 1.5 μL of sample solutions were taken at specific time intervals to measure the optical absorption of the sample solution. Background correction of the absorption spectrum was made with ultrapure water. All measurements were performed in triplicate (n=3) at room temperature and a relative humidity of ˜40%. ACF, TMZ, and PT2385 were measured at the wavelength of 325, 445, and 196 nm, respectively. When they are simultaneously released from 3-drug NanoMesh samples, the ACF and TMZ signals are distinct and not affected by the other drugs, while the absorption peak intensity of PT2385 at 196 nm has some overlap with that of the other two drugs. Therefore, based on the intensity ratio between 196 nm and the peak wavelengths of ACF and TMZ, the absorption intensity values of ACF and TMZ at 196 nm were calculated and then subtracted from the measured intensity at 196 nm to obtain the intensity at 196 nm of PT2385 drug.

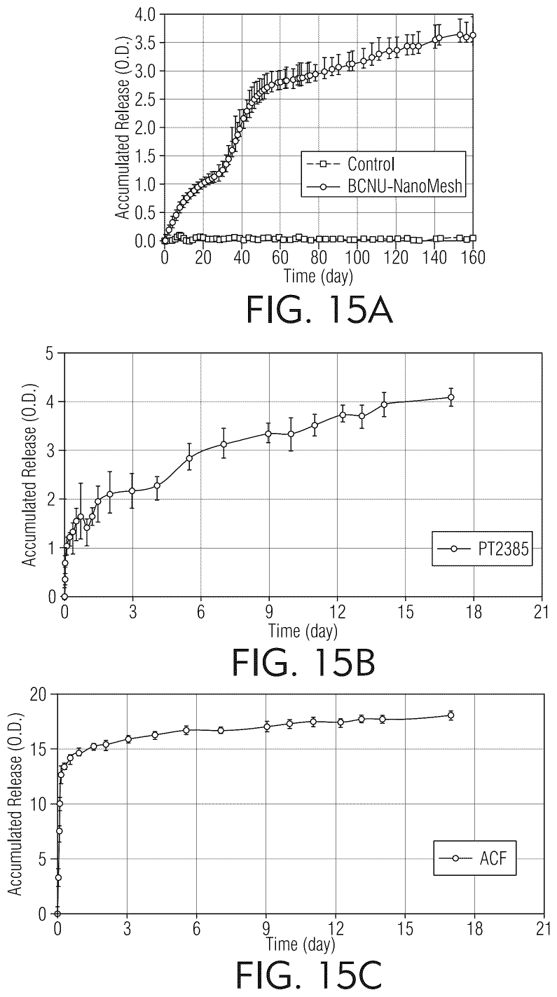

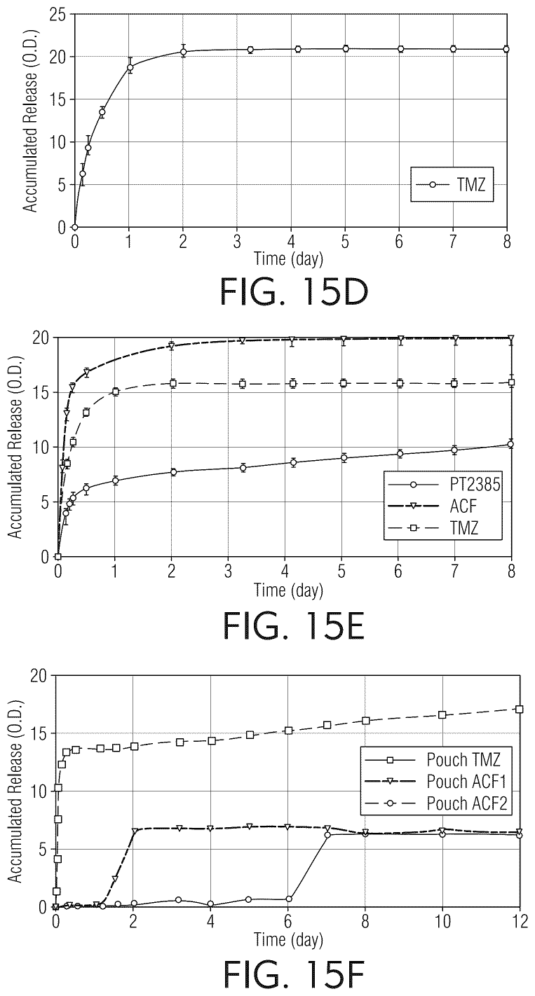

The drug release kinetics of the electrospun nanofiber NanoMeshes are shown in FIGS. 15A-15F with time on the x-axis and accumulated drug release (optical density) on the y-axis ((FIG. 15A) BCNU; (FIG. 15B) PT2385 20%; (FIG. 15C) ACF 10%; (FIG. 15D) TMZ 20%; (FIG. 15E) 3 Drugs (PT2385/ACF/TMZ); (FIG. 15F) NanoPouch with TMZ membrane (outer layer) & ACF inner NanoMesh (core layers)). It can be seen from FIG. 15F that this NanoPouch drug delivery device of the present disclosure enables timed release of TMZ and ACF on different schedules. The release profiles of ACF and TMZ in the 3-drug NanoMesh are very similar to those in the 1-drug profiles, whereas the PT2385 release kinetics were markedly different between the single and 3-drug NanoMesh. The PT2385 as a single drug was released continuously for 3 weeks, with a slow release rate (2.4 μg/day for 21 days) and the total amount released was also relatively low ˜0.057 mg for a period of 60 days. However, in the 3-Drug NanoMesh the kinetics data for PT2385 indicate a sustained release profile >9 weeks with a faster rate (53 μg/day for 20 days) and a ˜5 times higher total amount released (0.267 mg). A possible explanation could be an entraining effect due to the release of the other 2 drugs (ACF and TMZ) in the 3-drug NanoMesh. Co-incorporation of ACF and TMZ may affect the release rate of PT2385 in 3-Drug NanoMesh. In FIG. 15F, ACF2 has a thicker intermediate barrier layer than ACF1, so it obtained longer delay to be released from the core part.

### Example 2. In Vitro Cytotoxicity Measurements

GL261-LUC cell lines were cultured at 37° C. and 5% CO₂ using Dulbecco's Modified Eagle Medium (DMEM) with high glucose media, supplemented with 10% Fetal Bovine Serum (Sigma-Aldrich) and 1% Penicillin/Streptomycin.

Cell Viability Assays: Cell viability will be assessed using Cell Counting Kit 8 (CCK-8) (Dojindo Molecular Technologies, Japan). 2×10³ cells will be seeded on a 96-well plate and treated with various drug-containing nanofibers (ACF, PT2385, ACF-PT2385, ACF-PT2385-TMZ) or blank nanofibers after 24 h. CCK-8 reagent will be added after 48 h and cell viability quantified. The data will be normalized against untreated controls.

Statistical Analysis: In vitro cytotoxicity studies among treatment groups and the control group will be measured by one-way ANOVA with a post hoc Tukey test (a=0.05). The in vitro cytotoxicity of ACF, PT2385, ACF-PT2385 and ACF-PT2385-TMZ with radiation will be compared by two-tailed t-test, and significance will be evaluated at a=0.05. Appropriate positive and negative controls and statistical analysis will be used in each study to ensure scientific rigor.

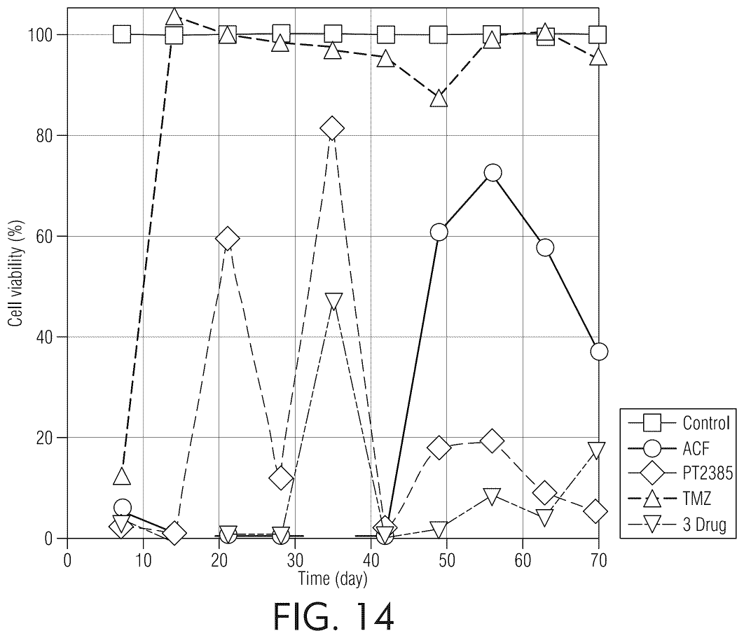

The cell viability results for the control, ACF 10%, PT2385 20%, and 3 Drugs (PT2385/ACF/TMZ) electrospun nanofiber NanoMesh are presented in FIG. 14. As can be seen, the TMZ-only NanoMesh shows no cytotoxicity after one week. The ACF-only NanoMesh shows ˜30-40% cytotoxicity from the seventh week on. The PT2385-only NanoMesh shows better cytotoxicity than the ACF-only membrane from the seventh week on. Significantly, the 3 Drug NanoMesh shows the highest long-term cytotoxicity. FIG. 14 shows a preliminary evaluation of the long-term cytotoxicity of drug-incorporated NanoMesh tabs. Although these preliminary tests presented a large variation of cytotoxicity over time, the 3-Drug NanoMesh shows highest cytotoxicity during the test period of 10 weeks. Interestingly, PT2385-only shows stronger cytotoxicity over time, while ACF-only case shows the opposite trend. TMZ-only case presented the shortest effect of only up to 1 week.

### Example 3. Hydrophobic Polymer Coated Electrospun Nanofiber Membranes

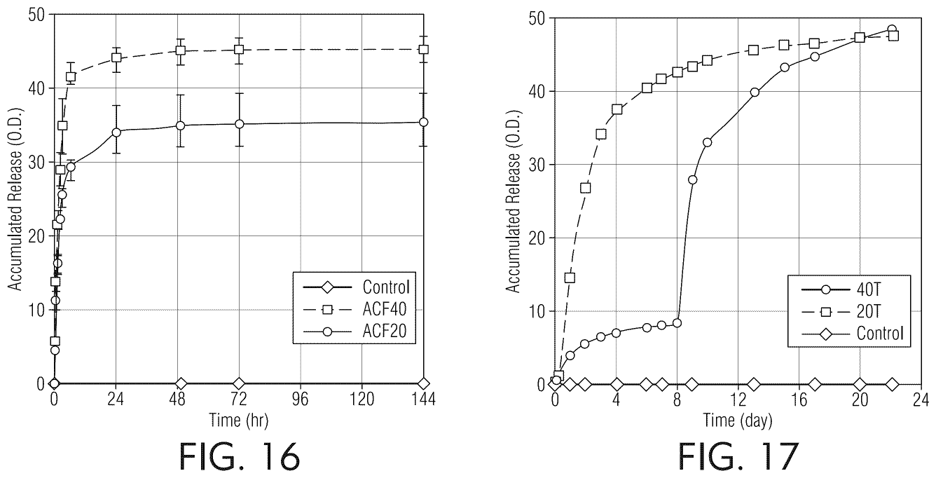

As discussed above, embodiments of the drug delivery devices of the present disclosure include coaxially electrospun nanofiber NanoMesh or dual-layer electrospun nanofiber NanoMesh wherein the perimeter edge, i.e., the sidewall, is coated with a hydrophobic polymer coating (see FIGS. 9A and 9B). To investigate the influence of the hydrophobic polymer coating, the drug release kinetics of four additional electrospun nanofiber NanoMesh were measured: (1) a coaxially electrospun nanofiber NanoMesh comprising 20 wt % ACF (“ACF20”; FIG. 16); (2) a Teflon-coated coaxially electrospun nanofiber NanoMeshes comprising 20 wt % ACF (“20T”: FIG. 17); (3) a coaxially electrospun nanofiber NanoMesh comprising 40 wt % ACF (“ACF40”; FIG. 16); and (4) a Teflon-coated dual-layer electrospun nanofiber NanoMeshes comprising 40 wt % ACF (“40T”; FIG. 17). Each of samples ACF20, 20T, ACF40, and 40T included between 24 and 32 electrospun nanofiber membrane layers. The Teflon coating as applied to the sidewalls of the NanoMeshes by twice applying a Teflon AF solution (1 wt % Teflon in Fluorinet's FC-75) to the sidewalls using a cotton tip. The ACF weight percentages provided for samples ACF20, 20T, ACF40, and 40T is with respect to the total weight of the electrospun nanofiber NanoMesh (i.e., with respect to the whole tab).

FIG. 16 is a chart showing the drug release kinetics for the non-coated coaxially electrospun nanofiber NanoMeshes ACF20 and ACF40. FIG. 17 is a chart showing the drug release kinetics for the Teflon-coated coaxially electrospun nanofiber NanoMeshes 20T and 40T. It was found ACF release may be sustained by roughly 30 times with the Teflon coating on the sidewall. For example, after 6 hrs of the ACF release, the accumulated absorption intensities of released-ACF20 and -ACF40 are 29 and 42 O.D., respectively, without the Teflon coating on the sidewall, while they are 1.36 and 1.0, respectively, with the Teflon coating on the sidewall. This indicates that the Teflon coating reduced the drug release rate˜21 and 42 times, respectively. Moreover, as can be seen from FIG. 17, the Teflon-coated dual-layer electrospun nanofiber NanoMesh 40T exhibits a significantly reduced release rate relative to Teflon-coated coaxially electrospun nanofiber NanoMesh 20T, despite these NanoMesh having similar ACF content due to the alternating layers in the 40T NanoMesh. Without wishing to be bound by theory, this reduced release rate is believed to be a result of the alternating hydrophobic intermediate layers within the stack forming the dual-layer electrospun nanofiber NanoMesh.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. The term “substantially” is used herein also to represent the degree by which a quantitative representation may vary from a stated reference without resulting in a change in the basic function of the subject matter at issue. Thus, it is used to represent the inherent degree of uncertainty that may be attributed to any quantitative comparison, value, measurement, or other representation, referring to an arrangement of elements or features that, while in theory would be expected to exhibit exact correspondence or behavior, may in practice embody something less than exact.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that where a first component is described as “comprising” or “including” a second component, it is contemplated that, in some embodiments, the first component “consists” or “consists essentially of” the second component. Additionally, the term “consisting essentially of” is used in this disclosure to refer to quantitative values that do not materially affect the basic and novel characteristic(s) of the disclosure.

It should be understood that any two quantitative values assigned to a property or measurement may constitute a range of that property or measurement, and all combinations of ranges formed from all stated quantitative values of a given property or measurement are contemplated in this disclosure.

Patents, applications, and publications mentioned in the specification are indicative of the levels of those skilled in the art to which the invention pertains. These patents and publications are incorporated herein by reference to the same extent as if each individual application or publication was specifically and individually incorporated herein by reference.

While particular embodiments have been illustrated and described herein, it should be understood that various other changes and modifications may be made without departing from scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A drug delivery device comprising:
a first layer comprising a first coaxially electrospun nanofiber membrane;
a second layer comprising a second coaxially electrospun nanofiber membrane;
a first therapeutic agent integrated into the first coaxially electrospun nanofiber membrane; and
a second therapeutic agent integrated into the second coaxially electrospun nanofiber membrane, the second therapeutic agent being different from the first therapeutic agent.

2. The drug delivery device of claim 1, wherein the first and second therapeutic agents are anti-neoplastic drugs.

3. The drug delivery device of claim 1, wherein the first therapeutic agent is configured to inhibit a first hypoxia-inducible factor and the second therapeutic agent is an alkylating agent.

4. The drug delivery device of claim 3, further comprising a third therapeutic agent integrated into the first coaxially electrospun nanofiber membrane and configured to inhibit a second hypoxia-inducible factor different from the first hypoxia-inducible factor.

5. The drug delivery device of claim 4, wherein:
the first hypoxia-inducible factor is HIF-1α; and
the second hypoxia-inducible factor is HIF-2α.

6. The drug delivery device of claim 5, wherein:
the first therapeutic agent is acriflavine (ACF);
the third therapeutic agent is PT2385; and
the alkylating agent is temozolomide (TMZ).

7. The drug delivery device of claim 1, wherein:
the first coaxially electrospun nanofiber membrane is folded and compressed to form a first coaxially electrospun nanofiber NanoMesh;
the second coaxially electrospun nanofiber membrane is folded and compressed to form a second coaxially electrospun nanofiber NanoMesh;
the first layer comprises a first disc that has been laser cut from the first coaxially electrospun nanofiber NanoMesh; and
the second layer comprises a second disc that has been laser cut from the second coaxially electrospun nanofiber NanoMesh.

8. The drug delivery device of claim 1, further comprising a third layer comprising a third coaxially electrospun nanofiber membrane, wherein the second therapeutic agent is also incorporated into the third coaxially electrospun nanofiber membrane, and wherein:
the second layer is positioned outward from a first surface of the first layer; and
the third layer is positioned outward from a second surface of the first layer, wherein the second surface of the first layer is opposite the first surface of the first layer.

9. The drug delivery device of claim 8, further comprising:
a first intermediate layer interposed between the first layer and the second layer; and
a second intermediate layer interposed between the first layer and the third layer.

10. The drug delivery device of claim 9, wherein the first and second intermediate layers are hydrophobic.

11. The drug delivery device of claim 9, wherein each of the first and second intermediate layers comprises an electrospun nanofiber membrane.

12. The drug delivery device of claim 9, further comprising a third therapeutic agent integrated into the first coaxially electrospun nanofiber membrane, wherein:
the first therapeutic agent is configured to inhibit a first hypoxia-inducible factor; and
the third therapeutic agent is configured to inhibit a second hypoxia-inducible factor different from the first hypoxia-inducible factor.

13. The drug delivery device of claim 12, wherein:
the first hypoxia-inducible factor is HIF-1α;
the second hypoxia-inducible factor is HIF-2α; and
the second therapeutic agent is an alkylating agent.

14. The drug delivery device of claim 13, wherein:
the first therapeutic agent is acriflavine (ACF);
the third therapeutic agent is PT2385; and
the alkylating agent is temozolomide (TMZ).

15. The drug delivery device of claim 12, wherein:
the first coaxially electrospun nanofiber membrane is folded and compressed to form a first coaxially electrospun nanofiber NanoMesh;
the second coaxially electrospun nanofiber membrane is folded and compressed to form a second coaxially electrospun nanofiber NanoMesh;
the third coaxially electrospun nanofiber membrane is folded and compressed to form a third coaxially electrospun nanofiber NanoMesh;
the first layer comprises a first disc that has been laser cut from the first coaxially electrospun nanofiber NanoMesh;
the second layer comprises a second disc that has been laser cut from the second coaxially electrospun nanofiber NanoMesh; and
the third layer comprises a third disc that has been laser cut from the third coaxially electrospun nanofiber NanoMesh.

16. The drug delivery device of claim 15, wherein:
the first hypoxia-inducible factor is HIF-1α;
the second hypoxia-inducible factor is HIF-2α; and
the second therapeutic agent is an alkylating agent.

17. The drug delivery device of claim 16, wherein:
the first therapeutic agent is acriflavine (ACF);
the third therapeutic agent is PT2385; and
the alkylating agent is temozolomide (TMZ).

18. A method for treating cancer in a subject, the method comprising:
delivering at least the first therapeutic agent and the second therapeutic agent to a target region of the subject by implanting the drug delivery device of claim 2 at or near the target region.

19. The method of claim 18, wherein the method is used to treat glioblastoma multiforme (GBM).

20. A method of manufacturing a drug delivery device, the method comprising:
preparing a first coaxially electrospun nanofiber membrane, wherein a first therapeutic agent is integrated into the first coaxially electrospun nanofiber membrane;
preparing a second coaxially electrospun nanofiber membrane, wherein a second therapeutic agent is integrated into the second coaxially electrospun nanofiber membrane;
folding and compressing the first coaxially electrospun nanofiber membrane to form a first coaxially electrospun nanofiber NanoMesh;
folding and compressing the second coaxially electrospun nanofiber membrane to form a second coaxially electrospun nanofiber NanoMesh;
laser cutting a first disc from the first coaxially electrospun nanofiber NanoMesh;
laser cutting a second disc from the second coaxially electrospun nanofiber NanoMesh; and
stacking the first and second discs.

21. The method of claim 20, further comprising pressing the first and second discs together using a heated stamp.

22. The method of claim 21, wherein prior to pressing the first and second discs together, the heated stamp is heated to a sealing temperature greater than or equal to 60° C. and less than or equal to 120° C.

23. The method of claim 20, further comprising:
preparing a third coaxially electrospun nanofiber membrane, wherein the second therapeutic agent is also integrated into the third coaxially electrospun nanofiber membrane folding and compressing the third coaxially electrospun nanofiber membrane to form a third coaxially electrospun nanofiber NanoMesh;
laser cutting a third disc from the third coaxially electrospun nanofiber NanoMesh;
stacking the first, second, and third discs such that:
the second disc is positioned outward from a first surface of the first disc; and
the third disc is positioned outward from a second surface of the first disc, wherein the second surface of the first disc is opposite the first surface of the first disc.

24. The method of claim 23, wherein:
a first intermediate layer is positioned between the first disc and the second disc; and
a second intermediate layer is positioned between the first disc and the third disc.

25. The method of claim 24, further comprising pressing together, in this order, the second disc, the first intermediate layer, the first disc, the second intermediate layer, and the third disc, using a heated stamp that has been heated to a sealing temperature greater than or equal to 70° C. and less than or equal to 120° C.

26. A drug delivery device comprising:
a coaxially electrospun nanofiber membrane that is folded and compressed to form a coaxially electrospun nanofiber NanoMesh comprising:
a first surface;
a second surface opposite the first surface; and
a sidewall extending around a perimeter of the coaxially electrospun nanofiber NanoMesh between the first surface and the second surface, wherein the sidewall is coated with a hydrophobic polymer coating; and
a first therapeutic agent integrated into the coaxially electrospun nanofiber membrane.

27. The drug delivery device of claim 26, wherein the hydrophobic polymer coating comprises Teflon.

28. The drug delivery device of claim 26, wherein the sidewall is coated twice with the hydrophobic polymer coating.

29. The drug delivery device of claim 26, wherein the coaxially electrospun nanofiber NanoMesh comprises:
the coaxially electrospun nanofiber membrane; and
a homogeneously electrospun nanofiber membrane.

30. The drug delivery device of claim 29, wherein the homogeneously electrospun nanofiber membrane is hydrophobic and substantially drug-free.

31. The drug delivery device of claim 30, wherein the coaxially electrospun nanofiber membrane and the homogeneously electrospun nanofiber membrane are arranged in an altemating fashion through a thickness of the drug delivery device.
